(19) United States
(12) Patent Application Publication
(10) Pub. No.: US 2026/0284038 A1
(43) Pub. Date: Sep. 24, 2026

(54) TREATMENT OF PRURIGO NODULARIS

(71) Applicant: Trevi Therapeutics, Inc., New Haven, CT (US)

(72) Inventors: Thomas SCIASCIA, Brookline, NH (US); Jennifer L. GOOD, Sandy Hook, CT (US); Amale HAWI, Ridgefield, CT (US)

(21) Appl. No.: 19/669,004

(22) Filed: May 6, 2026

Related U.S. Application Data
(63) Continuation of application No. 19/331,819, filed on Sep. 17, 2025, now abandoned.
Application No. 19/331,819 is a continuation of application No. 19/028,062, filed on Jan. 17, 2025, now abandoned.
Application No. 19/028,062 is a continuation of application No. 18/342,395, filed on Jun. 27, 2023, now abandoned.
Application No. 18/342,395 is a continuation of application No. 17/545,487, filed on Dec. 8, 2021, now abandoned.
Application No. 17/545,487 is a continuation of application No. 17/313,409, filed on May 6, 2021, now abandoned.
Application No. 17/313,409 is a continuation of application No. 17/038,922, filed on Sep. 30, 2020, now abandoned.
Application No. 17/038,922 is a continuation of application No. 16/786,733, filed on Feb. 10, 2020, now abandoned.
Application No. 16/786,733 is a continuation of application No. 16/453,586, filed on Jun. 26, 2019, now abandoned.
Application No. 16/453,586 is a continuation of application No. 15/793,153, filed on Oct. 25, 2017, now abandoned.
(60) Provisional application No. 62/412,578, filed on Oct. 25, 2016.

(51) Int. Cl. A61K 31/485; A61K 9/00; A61K 45/06; A61P 17/04

(52) U.S. Cl. CPC A61K 31/485; A61K 9/0053; A61P 17/04; A61K 45/06

(57) ABSTRACT

The present invention relates to methods for treating prurigo nodularis with anti-pruritic compositions, wherein the method provides a therapeutic effect in a patient.

### CROSS-REFERENCE TO RELATED APPLICATIONS

The present application is a continuation of U.S. application Ser. No. 19/331,819, filed Sep. 17, 2025, which is a continuation of U.S. application Ser. No. 19/028,062, filed Jan. 17, 2025, which is a continuation of U.S. application Ser. No. 18/342,395, filed Jun. 27, 2023, which is a continuation of U.S. application Ser. No. 17/545,487, filed Dec. 8, 2021, which is a continuation of U.S. application Ser. No. 17/313,409, filed May 6, 2021, which is a continuation of U.S. application Ser. No. 17/038,922, filed Sep. 30, 2020, which is a continuation of U.S. application Ser. No. 16/786,733, filed Feb. 10, 2020, which is a continuation of U.S. application Ser. No. 16/453,586, filed Jun. 26, 2019, which is a continuation of U.S. application Ser. No. 15/793,153, filed Oct. 25, 2017, which claims the benefit of priority to U.S. Provisional Application No. 62/412,578, filed on Oct. 25, 2016, the contents of each of which are hereby incorporated by reference in their entirety.

### FIELD OF THE INVENTION

The present invention relates to methods for treating prurigo nodularis in patients using nalbuphine compositions.

### BACKGROUND

Pruritus, or itch, is a sensation that stimulates the desire to scratch. Pruritus can be either generalized to multiple non-contiguous anatomical areas or localized to one specific anatomical area over the body skin surface. The cause of pruritus is not fully understood. Proposed contributors to the pathogenesis of pruritus may include anemia or other manifestation of erythropoietin deficiency, histamine release from skin mast cells, skin dryness, secondary hyperparathyroidism, hyperphosphatemia with increased calcium phosphate deposition in the skin and alterations in the endogenous opioidergic system with overexpression of opioid μ-receptors.

Prurigo Nodularis (PN) is an intensely pruritic dermatologic condition with the presence of pruriginous skin lesions of papules as well as nodules with excoriations and ulcerations. In terms of treatment options for PN, there have been a variety of medical interventions discussed and an effective treatment is still needed.

### SUMMARY OF THE INVENTION

The present invention, among other things, provides methods of treating pruritus comprising administering an effective amount of an anti-pruritus agent to a patient in need of such treatment. In some embodiments, the anti-pruritus agent is nalbuphine or a pharmaceutically acceptable salt, solvate or ester thereof.

In some embodiments, the patient in need of a treatment of pruritus is a patient with prurigo nodularis. In certain embodiment, the patient has moderate or severe prurigo nodularis.

According to some embodiments of the present invention, the method of treating prurigo nodularis comprises administering for at least a week to a patient in need thereof a daily dose of at least about 180 mg of nalbuphine or a pharmaceutically acceptable salt, solvate or ester thereof. In some embodiments, the method of treating prurigo nodularis comprises administering for at least a week to a patient in need thereof a daily dose of at least about 360 mg of nalbuphine or a pharmaceutically acceptable salt, solvate or ester thereof. In some embodiments, about 90 mg of the anti-pruritus agent is administered twice a day. In some embodiments, about 180 mg of the anti-pruritus agent is administered once a day. In some embodiments, about 180 mg of the anti-pruritus agent is administered twice a day. In some embodiments, about 360 mg of the anti-pruritus agent is administered once a day.

In some embodiments, the anti-pruritus agent is administered for about 8 weeks. In some embodiments, the anti-pruritus agent is administered for about 10 weeks. In some embodiments, the anti-pruritus agent is administered for about 12 weeks. In some embodiments, the anti-pruritus agent is administered for about 18 weeks. In some embodiments, the anti-pruritus agent is administered for about 50 weeks.

In some embodiments, after the treatment the patient experiences a substantial reduction in itch compared to prior to the treatment.

In some embodiments, the method of treating pruritus further includes a step of titrating the dose of the anti-pruritus agent for at least about one week until a steady state is achieved in the patient. In one embodiment, the titration is conducted for about 2 weeks until a steady state is achieved in the patient. In another embodiment, the titration is conducted for about 7 days to about 30 days until a steady state is achieved in the patient. In another embodiment, the titration is conducted for about 12 days to about 20 days until a steady state is achieved in the patient.

In certain embodiments, ascending doses of the anti-pruritus agent are administered during the titration until a steady state is achieved in the patient. In certain embodiments, ascending doses of the anti-pruritus agent are administered during the titration until an effective amount of 90 mg or 180 mg is achieved in the patient.

In one embodiment, the titration is initiated with a dose of about 15 mg once or twice a day. In another embodiment, the titration is initiated with a dose of about 30 mg once or twice a day. In certain embodiments, the titration comprises administering the anti-pruritus agent in increments ranging from about 15 mg to about 30 mg. In certain embodiments, the titration comprises administering the anti-pruritus agent in increments ranging from about 15 mg to about 60 mg. In certain embodiments, titration twice a day is with an AM dosage and a PM dosage, wherein the PM dosage is higher than or the same as the AM dosage.

In accordance with some embodiments of the present invention, the rate of adverse events after the treatment with the anti-pruritus agent is substantially the same as the rate of adverse events after administering a placebo for the same period of time.

According to some embodiments of the present invention, clinical studies show that subjects treated with an anti-pruritus agent experience a statistically significant reduction of itch compared to subjects treated with a placebo. In some embodiments, the statistically significant reduction of itch is indicated by a p value of less than or equal to about 0.05. In some embodiments, the patient with moderate or severe baseline itch prior to the treatment experiences mild itch after the treatment.

According to some embodiments of the present invention, after the treatment the patient experiences a reduction of itch that is characterized by at least about a 30%, 40%, or 50% decline in worst itch intensity Numerical Rating Scale (NRS) value. In some embodiments, after the treatment the patient experiences a reduction of itch that is characterized by at least about a 30%, 40%, or 50% decline in average itch intensity Numerical Rating Scale (NRS) value.

According to some embodiments of the present invention, after the treatment the patient experiences a reduction of itch that is characterized by at least about a 10%, 20%, 30%, 40%, or 50% decline in intensity of the itchy Verbal Rating Scale (VRS) value. In some embodiments, after the treatment the patient experiences a reduction of burning sensation that is characterized by at least about a 10%, 20%, 30%, 40%, or 50% decline in intensity of the burning Verbal Rating Scale (VRS) value. In some embodiments, after the treatment the patient experiences a reduction of stinging sensation that is characterized by at least about a 10%, 20%, 30%, 40%, or 50% decline in intensity of the stinging Verbal Rating Scale (VRS) value.

According to some embodiments of the present invention, after the treatment the patient experiences a reduction of itch that is characterized by at least about a 10%, 20%, or 30% improvement in Itchy Quality of Life (ItchyQoL) total scale score or in any of the respective subscales of: Symptom Subscale score, Functional Subscale score, or Emotion Subscale score.

According to some embodiments of the present invention, after the treatment the patient experiences a reduction of itch that is characterized by at least about a 10%, 20%, or 30% improvement in Patient Benefit Index-pruritus version (PBI-P) scale.

According to some embodiments of the present invention, after the treatment the patient experiences a reduction of itch that is characterized by at least one category/stage improvement in Prurigo Activity Score (PAS) domains of number of prurigo lesions, prurigo lesions with excoriations/crusts and/or healed prurigo lesions.

In accordance with some embodiments of the present invention, the method of treating pruritus does not produce a substantial aquaretic effect. In accordance with some embodiments of the present invention, after the treatment the rate of muscoskeletal complaints of the patient is lower than that of the patient prior to the treatment.

In some embodiments, the method of treating pruritus further includes administering at least one additional antipruritic drug. In certain embodiments, at least one additional antipruritic drug is selected from the group consisting of antihistamines (for example, loratadine), corticosteroids (for example, prednisone), capsaicin, calcineurin inhibitors (for example, tacrolimus), antibiotics (for example, tetracycline), anti-convulsants (for example, gabapentin), immunosupressants (for example, methotrexate), anti-depressants (for example, amitriptyline), neuroleptics (for example, clozapine), benzodiazepine (for example, diazepam), immunomodulators (for example, thalidomide) or with the addition of non-pharmacologic treatment such as ultraviolet radiation therapy.

In some embodiments, the anti-pruritus agent is in the form of an extended release oral dosage form.

In some embodiments, the anti-pruritus agent is administered in a formulation comprising nalbuphine hydrochloride, mannitol, hydroxypropyl cellulose, locust bean gum, xanthan gum, calcium sulfate dihydrate, and magnesium stearate.

The present methods, and advantages thereof, are further illustrated by the following non-limiting detailed description, including the Examples.

### BRIEF DESCRIPTION OF THE DRAWINGS

FIG. 1 illustrates the Worst Itching Intensity Numerical Rating Scale (NRS).

FIG. 2 is a schematic overview of the screening and treatment regimens of three randomized groups of patients. NAL 180=nalbuphine ER tablets 180 mg BID; NAL 90=nalbuphine ER tablets 90 mg BID; and placebo BID.

FIG. 3 is a graphical representation of Mean Change from Baseline to the Last Observation in the Worst Itching Numerical Rating Scale (range, 0-10; anchors at 0 “no itching”; 4-6 “moderate itching”; and 10 “worst possible itching”) for all MITT patients (N=62, at left) and for completing patients (N=50, at right). NAL 180=nalbuphine ER tablets 180 mg BID; NAL 90=nalbuphine ER tablets 90 mg BID of Example 2.

FIG. 4 is a graphical representation of the Proportion of Patients with Percentage Reduction in 7-Day Worst Itch Intensity from Baseline to Last Observed Visit by Treatment Group (Modified Intent-to-Treat Population). NAL 180=nalbuphine ER tablets 180 mg BID; NAL 90=nalbuphine ER tablets 90 mg BID of Example 2.

FIG. 5 is a graphical representation of the Proportion of Patients with Percentage Reduction in 7-Day Worst Itch Intensity from Baseline to Week 10 for Completers by Treatment Group (Modified Intent-to-treat Population). NAL 180=nalbuphine ER tablets 180 mg BID; NAL 90=nalbuphine ER tablets 90 mg BID of Example 2.

FIG. 6 is a graphical representation of the total ItchyQOL score by week for (1) patients in the NAL 180 treatment group and (2) patients in the placebo group. NAL 180=nalbuphine ER tablets 180 mg BID of Example 2.

FIG. 7 is an overall schematic of a Phase 2 extension study (TR03ext) described in Example 3.

FIG. 8A shows the pruriginous lesions of a patient from Example 1 at baseline and FIG. 8B shows the healed pruriginous lesions of the same patient at Week 50 in the extension study described in Example 3 (TR03ext).

### DEFINITIONS

The term “about” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The terms “administer,” “administering” or “administration” as used herein refer to either directly administering a compound or pharmaceutically acceptable salt or ester of the compound or a composition comprising the compound or pharmaceutically acceptable salt or ester of the compound to a patient.

The term “adverse event” (AE) as used herein is defined as any untoward medical occurrence in a clinical investigation patient reported on or after the first screening date. An AE does not necessarily have to have a causal relationship with the treatment. An AE can therefore be any unfavorable and unintended sign (including an abnormal laboratory finding), symptom whether or not related to the medicinal (investigational) product, or disease temporally associated with the use of a medicinal (investigational) product. Typical adverse events include nausea, vomiting, somnolence, dizziness and hallucination. In accordance with the present invention, the rate of adverse events after the treatment is substantially the same as the rate of adverse events after administering a placebo for the same period of time.

The term “carrier” as used herein encompasses carriers, excipients, and diluents, meaning a material, composition or vehicle, such as a liquid or solid filler, diluent, excipient, solvent or encapsulating material involved in carrying or transporting a pharmaceutical agent from one organ, or portion of the body, to another organ or portion of the body.

The term “disorder” is used in this disclosure to mean, and is used interchangeably with, the terms disease, condition, or illness, unless otherwise indicated.

The terms “effective amount” and “therapeutically effective amount” are used interchangeably in this disclosure and refer to an amount of a compound, or a salt, solvate or ester thereof, that, when administered to a patient, is capable of performing the intended result. For example, an effective amount of an anti-pruritic agent is that amount which is required to reduce at least one symptom of pruritus in a patient, e.g. the amount required to reduce the itching sensation in a patient. The actual amount which comprises the “effective amount” or “therapeutically effective amount” will vary depending on a number of conditions including, but not limited to, the severity of the disorder, the size and health of the patient, and the route of administration. A skilled medical practitioner can readily determine the appropriate amount using methods known in the medical arts.

The phrase “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “salts” as used herein embraces pharmaceutically acceptable salts commonly used to form alkali metal salts of free acids and to form addition salts of free bases. The nature of the salt is not critical, provided that it is pharmaceutically acceptable. The term “salts” also includes solvates of addition salts, such as hydrates, as well as polymorphs of addition salts. Suitable pharmaceutically acceptable acid addition salts can be prepared from an inorganic acid or from an organic acid. Examples of such inorganic acids are hydrochloric, hydrobromic, hydroiodic, nitric, carbonic, sulfuric, and phosphoric acid. Appropriate organic acids can be selected from aliphatic, cycloaliphatic, aromatic, arylaliphatic, and heterocyclyl containing carboxylic acids and sulfonic acids, for example formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, cyclohexylaminosulfonic, algenic, 3-hydroxybutyric, galactaric and galacturonic acid.

The term “treating” as used herein with regard to a patient, refers to improving at least one symptom of the patient's disorder. Treating can be curing, improving, or at least partially ameliorating a disorder.

The term “therapeutic effect” as used herein refers to a desired or beneficial effect provided by the method and/or the composition. For example, the method for treating pruritus provides a therapeutic effect when the method reduces at least one symptom of pruritus, e.g., itching sensation, in a patient.

### DETAILED DESCRIPTION

According to the present invention, pruritus includes any itchy or pruritic condition, e.g., a sensation that causes the desire to scratch. Prurigo is a pruritic condition that is characterized by any type of pruriginous lesion (such as papular, nodular, plaque and umbilicated lesions) induced by scratching due to chronic pruritus. Pruriginous lesions include excoriated, scaling, and/or crusted papules, nodules and plaques, often with a whitish or pink center and hyper-pigmented border.

The European Academy of Dermatology and Venereology (EADV) Task Force on Pruritus (TFP) consensus statement (Pereira, M P et al (2017), “European academy of dermatology and venereology European prurigo project: expert consensus on the definition, classification and terminology of chronic prurigo.” J Eur Acad Dermatol Venereol. 2017, doi:10.1111/jdv.14570) summarizes the most up to date medical and scientific data on PN. The TFP discussed the etiology and diagnosis of prurigo nodularis. The Task Force on Pruritus recommended that the term of “chronic prurigo” be the diagnostic term used by medical practitioners and that the diagnosis of “chronic prurigo” encompass all of the variants of pruriginous lesions, such as papular, nodular, plaque and umbilicated prurigo and any other prurigo skin manifestations. Thus, prurigo nodularis and chronic prurigo can be used interchangeably as terminology for the same clinical condition.

Currently, there is no known biomarker that explains the pathophysiology of chronic prurigo. Instead, chronic prurigo is clinically diagnosed by observations that are independent of the etiology of the underlying pruritus. The diagnostic clinical symptoms of chronic prurigo include the presence of chronic pruritus (≥6 weeks), a history and/or signs of repeated scratching (for example, excoriations, scars) and the localized or generalized presence of multiple pruriginous lesions.

The etiology or predisposing factors leading to the development of pruriginous lesions of chronic prurigo are largely unknown (see, Eigelshoven S, et al. “Prurigo nodularis” CME Dermatol. 2009; 4(3): 140-55; “Prurigo nodularis: a benign dermatosis derived from a persistent pruritis”, Acta Dermatovenerol Croat. 2008; 16(1):38-44; and Schwartz 2008; and Lee M R, et al. “Prurigo nodularis: a review”, Australas J Dermatol. 2005; 46(4):211-20.). The hypothesis for the etiology of PN as being a reaction pattern due to a “vicious cycle of repeated itching and scratching” has gained wider acceptance in the medical community (Iking A, et al. “Prurigo as a symptom of atopic and non-atopic diseases: aetiological survey in a consecutive cohort of 108 patients” J Eur Acad Dermatol Venereol. 2013; 27(5):550-7). There are currently no FDA-approved therapies for treating prurigo nodularis.

In one aspect, the present invention provides a method of treating pruritus comprising administering an effective amount of an anti-pruritus agent for at least about a week to a patient in need of such treatment, wherein the anti-pruritus agent is nalbuphine or a pharmaceutically acceptable salt, solvate or ester thereof. In accordance with some embodiments of the present invention, at least about 90 mg or 180 mg of the anti-pruritus agent is administered. In some embodiments, about 90 mg of the anti-pruritus agent is administered twice a day. In some embodiments, about 180 mg of the anti-pruritus agent is administered once a day. In some embodiments, about 180 mg of the anti-pruritus agent is administered twice a day. In some embodiments, about 360 mg of the anti-pruritus agent is administered once a day.

In another embodiment, methods of the present invention are used for the treatment of chronic prurigo. In certain embodiments, Nalbuphine HCl is used or indicated for the treatment of itch in adult patients with moderate to severe chronic prurigo.

In another embodiment, methods of the present invention are used for the treatment of prurigo nodularis. In certain embodiments, Nalbuphine HCl is used or indicated for the treatment of itch in adult patients with moderate to severe prurigo nodularis.

In accordance with some embodiments of the present invention, the method provides a therapeutic effect without producing a substantial adverse event. In some embodiments, the rate of adverse events after the treatment with the anti-pruritus agent is substantially the same as the rate of adverse events after administering a placebo for the same period of time.

In accordance with some embodiments of the present invention, the method of treating pruritus does not produce a substantial aquaretic effect.

In accordance with some embodiments of the present invention, the method of treating pruritus provides healing of pruriginous lesions such as nodules and papules. In some embodiments, scratching is reduced through treatment, and thus stinging, burning, itching and pain associated with pruriginous lesions such as the nodules, papules and lesions are reduced.

In accordance with some embodiments of the present invention, the method of treating prurigo nodularis provides healing of pruriginous lesions. In certain embodiments, method of treating prurigo nodularis provides healing of pruriginous lesions such as nodules, papules and/or plaques.

In accordance with some embodiments of the present invention, the method of treating prurigo nodularis provides for reduction in the amount of excoriated lesions or the total number of pruriginous lesions

### Nalbuphine

Nalbuphine as employed in the present methods can form a part of a pharmaceutical composition by combining nalbuphine, or a pharmaceutically acceptable salt, solvate or ester thereof, with a pharmaceutically acceptable carrier. Additionally, the compositions can include an additive selected from the group consisting of adjuvants, excipients, diluents, release-modifying agents and stabilizers. The composition can be an immediate release formulation, a delayed release formulation, a sustained release formulation or an extended release formulation.

Nalbuphine HCl (17-(cyclobutylmethyl)-4,5α-epoxymorphinian-3, 6α, 14-triol, hydrochloride) is a synthetic opioid. Structurally, nalbuphine is a derivative of 14 hydroxymorphine.

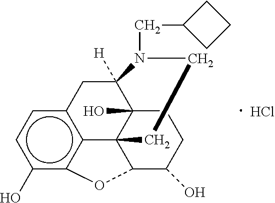

Nalbuphine HCl is currently available only as a generic medication in an injectable form. An injectable form of nalbuphine has been available as an approved drug formulation since 1978. Nubain® was the innovator brand injectable form of nalbuphine on which the presently sold generic bioequivalent injectable formulations are based. The injectable formulation is currently approved for use in the relief of moderate to severe pain, a supplement to balanced anesthesia, for pre-operative and post-operative analgesia and obstetrical analgesia during labor and delivery.

The present invention also includes pharmaceutically acceptable esters of the anti-pruritus agent. The term “ester” denotes a derivative of the agent containing an ester functional group (as described herein), which is capable of releasing the agent when the ester form is administered to a patient. Release of the active ingredient occurs in vivo. Pharmaceutically acceptable esters can be prepared by techniques known to one skilled in the art. These techniques generally modify appropriate functional groups in a given compound. These modified functional groups however regenerate original functional groups by metabolism of the compound in vivo. Esters include compounds wherein a hydroxy, carboxylic, or a similar group is modified.

Suitable pharmaceutically acceptable esters for a hydroxyl group include inorganic esters such as phosphate esters and α-acyloxyalkyl ethers and related compounds which, as a result of in vivo hydrolysis of the ester, provide the parent hydroxy group. In vivo hydrolyzable ester forming groups for hydroxy include alkanoyl (e.g., C₁₋₁₀ linear, branched or cyclic alkyl), benzoyl, phenylacetyl and substituted benzoyl and phenylacetyl, alkoxycarbonyl (to give alkyl carbonate esters), dialkylcarbamoyl and N—(N, N-dialkylaminoethyl)-N-alkylcarbamoyl (to give carbamates), N, N-dialkylaminoacetyl and carboxyacetyl.

### Formulations

The methods of the present invention can employ various formulations for administration to patients, e.g., humans and animals in unit dosage forms, such as tablets, capsules, pills, powders, granules, sterile parenteral solutions or suspensions, and oral solutions or suspensions, and oil-water emulsions containing suitable quantities of an anti-pruritic agent, e.g., nalbuphine, or pharmaceutically acceptable salts or esters thereof.

Oral pharmaceutical dosage forms can be either solid or liquid. The solid dosage forms can be tablets, capsules, granules, and bulk powders. Types of oral tablets include compressed, chewable lozenges and tablets, which can be enteric-coated, sugar-coated or film-coated. Capsules can be hard or soft gelatin capsules, while granules and powders can be provided in non-effervescent or effervescent form with the combination of other ingredients known to those skilled in the art. In other embodiments, the oral dosage form may be an osmotic-controlled release oral delivery system (OROS). In other embodiments, the oral dosage form may include matrix-embedded dosage forms or related devices. In some embodiments, the present oral dosage forms may include orally-disintegrating tablets.

Pharmaceutically acceptable carriers utilized in tablets include binders, lubricants, diluents, disintegrating agents, coloring agents, flavoring agents, and wetting agents.

Liquid oral dosage forms include aqueous solutions, emulsions, suspensions, solutions and/or suspensions reconstituted from non-effervescent granules and effervescent preparations reconstituted from effervescent granules.

Aqueous solutions include, for example, elixirs and syrups. Emulsions can be either oil-in water or water-in-oil. Elixirs are clear, sweetened, hydroalcoholic preparations. Pharmaceutically acceptable carriers used in elixirs include solvents. Syrups can be concentrated aqueous solutions of a sugar, for example, sucrose, and can contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions can use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substance used in effervescent granules, to be reconstituted into a liquid oral dosage form, can include organic acids and a source of carbon dioxide. Coloring and flavoring agents can be used in all of the above dosage forms.

Parenteral administration of the formulations of the present invention includes intravenous, subcutaneous and intramuscular administrations of immediate, sustained (e.g., depot), extended, and/or modified release formulations (e.g., as described herein). Preparations for parenteral administration include sterile solutions ready for injection, sterile dry soluble products ready to be combined with a solvent just prior to use, including hypodermic tablets, sterile suspensions ready for injection, sterile dry insoluble products ready to be combined with a vehicle just prior to use and sterile emulsions. The solutions can be either aqueous or nonaqueous. Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

The concentration of the pharmaceutically active compound can be adjusted so that an injection provides an effective amount to produce the desired pharmacological effect. The exact dose depends on the age, weight and condition of the patient or animal, as is known in the art. The unit-dose parenteral preparations are packaged in an ampoule or a syringe with a needle. All preparations for parenteral administration must be sterile, as is known and practiced in the art. Illustratively, intravenous or intra-arterial infusion of a sterile aqueous solution containing an anti-pruritic agent is an effective mode of administration.

Pharmaceutical dosage forms for rectal administration can be rectal suppositories, capsules and tablets for systemic effect. Rectal suppositories as used herein mean solid bodies for insertion into the rectum which melt or soften at body temperature releasing the pharmacologically and/or therapeutically active ingredients contained in the composition of this invention. Pharmaceutically acceptable substances utilized in rectal suppositories are bases or vehicles and agents to raise the melting point. Examples of bases include cocoa butter (theobroma oil), glycerin-gelatin, carbowax, polyoxyethylene glycol and mixtures of mono-, di- and triglycerides of fatty acids. Combinations of the various bases can be used. Agents to raise the melting point of suppositories include spermaceti and wax. Rectal suppositories can be prepared either by the compressed method or by molding. The typical weight of a rectal suppository is about 2 to 3 gm. Tablets and capsules for rectal administration can be manufactured using the same pharmaceutically acceptable substance and by the same methods as for formulations for oral administration.

The compositions can be suspended in micronized or other suitable form or can be derivatized to produce a more soluble active product. The form of the resulting composition depends upon a number of factors, including the intended mode of administration and the solubility of the anti-pruritic agent in the selected carrier or vehicle. The effective concentration is sufficient for treating or alleviating pruritus, and can be empirically determined. The concentration is generally greater than the concentration for systemic administration of the compound.

The resulting mixture can be a solution, suspension, emulsion or the like, and can be formulated as a cream, gel, ointment, emulsion, solution, elixir, lotion, suspension, tincture, paste, foam, aerosol, irrigation, spray, suppository, bandage, or any other formulation suitable for topical or local administration. Modes of administration can include topical application to the skin, scalp, eyes, and/or nasal, buccal or sublingual mucosa.

Pharmaceutical and cosmetic carriers or vehicles suitable for administration of the compositions include any such carriers known to those skilled in the art to be suitable for the particular mode of administration. The anti-pruritic agent can be included in the carriers in amounts sufficient to exert a therapeutically useful effect without serious toxic effects on the treated individual.

To formulate these compositions, a weight fraction of an anti-pruritic agent is dissolved, suspended, dispersed or otherwise mixed in a selected vehicle at an effective concentration such that the pruritic condition is relieved or ameliorated. Generally, emollient or lubricating vehicles that help hydrate the skin are more preferred than volatile vehicles, such as ethanol, that dry the skin. Examples of suitable bases or vehicles for preparing compositions for use with human skin are petrolatum, petrolatum plus volatile silicones, lanolin, cold cream (USP), and hydrophilic ointment (USP).

The compositions employed in the present methods can relieve pruritus when applied to the skin. The composition can be administered topically to the affected area up to eight times per day, as needed, to provide reduction in and relief from itching. Relief can be temporary or permanent, and can even be evident after a single dose of the composition. When the composition is administered in a form other than a topical preparation, it should be administered in an amount sufficient to provide relief from pruritus that is within safety guidelines established by the FDA. Determining the appropriate amount to administer to a patient is within the skill of the person of ordinary skill in the art in association with teachings provided by the present invention.

Solutions of the compositions of this invention intended for topical administration contain an amount of the composition effective to deliver an anti-pruritic amount, typically at a concentration of between about 0.01% w/w to about 5% w/w. The balance of the solution is water, a suitable organic solvent or other suitable solvent or buffer. These compositions that are formulated as solutions or suspensions can be applied to the skin, or can be formulated as an aerosol or foam and applied to the skin as a spray-on. The aerosol compositions typically contain from 25% to 80% w/w, preferably from 30% to 50% w/w, of a suitable propellant. Gel compositions can be formulated by simply admixing a suitable thickening agent to the solution or suspension.

Solutions and suspensions can also be topically applied to the eyes and mucosa. Solutions, particularly those intended for ophthalmic use, can be formulated as 0.01%-10% w/w isotonic solutions, pH about 5-7, with appropriate salts, and preferably containing one or more of the compositions herein at a concentration of about 0.1% w/w, up to about 5% w/w or more. Suitable ophthalmic solutions are known in the art.

Compositions of solid forms intended for topical application can be formulated as stick-type compositions intended for application to the lips or other parts of the body. Such compositions contain an effective amount of an anti-pruritic agent, e.g. nalbuphine or a pharmaceutically acceptable salt, solvate or ester thereof. The amount of the anti-pruritic agent present is typically from about 0.01% w/w to about 5% w/w. The solids also contain from about 40% to 98% w/w, preferably from about 50% to 90% w/w, of emollients. This composition can further contain from 1% to 20% w/w, preferably from 5% to 15% w/w, of a suitable thickening agent, and, if desired or needed, emulsifiers and water or buffers.

In addition, the compositions, and preparations containing the compositions, can also be coated on bandages, mixed with bioadhesives, or included in dressings. Thus, combinations of bandages, bioadhesives, dressings and other such materials and the compositions formulated as described herein are provided.

### Sustained Release

Nalbuphine formulations that can be employed in the present methods include oral sustained release nalbuphine formulations as described in U.S. Provisional Pat. Appl. Nos. 60/772,466, 60/710,772, and 62/011,936; U.S. patent application Ser. No. 11/509,347 (published as US 2007/0048376), Ser. No. 12/154,496 (published as US 2009/0030026), and Ser. No. 14/738,550; and PCT Appl. No. PCT/US2015/035650; each of which is incorporated herein by reference in their entireties.

“Sustained release” or “extended release” means that the nalbuphine or pharmaceutically acceptable salt, solvate or ester thereof is released from the formulation at a controlled rate so that therapeutically beneficial blood levels (but below toxic levels) of the nalbuphine or pharmaceutically acceptable salt, solvate or ester thereof are maintained over an extended period of time. Alternatively, “sustained release” or “extended release” means that the desired pharmacologic effect is maintained over an extended period of time.

The half-life of nalbuphine injectable formulations (i.e., IV or IM or SC) has been reported to be relatively short, only about 2-3 hours. In some embodiments, the present methods can employ oral sustained release formulations of nalbuphine including an anti-pruritic effective amount of nalbuphine or a pharmaceutically acceptable salt, solvate or ester thereof. The oral sustained release formulations can provide a controlled release and a lower Cmax of the anti-pruritus agent over a longer period than observed for bolus injections or immediate release oral formulations (e.g., at least about 8-12 hours). Reducing the frequency of dosing provides the potential for enhanced patient convenience and compliance with the present methods. The lower dosing frequency also has the potential to provide reduced side effects because the patient may be exposed to lower peak concentrations of agent over time.

Without wishing to be bound by a particular theory, the longer than expected duration of anti-pruritic effect is attributed to the enterohepatic recirculation of nalbuphine. Nalbuphine forms a glucuronic acid or other type of conjugated metabolite in vivo through enzymatic reaction with an enzyme system such as UDP-glucuronyl transferase. It is also possible that enterohepatic recirculation also occurs when parent drug in the bile is released from the gallbladder into the intestine and reabsorbed. Once formed, the conjugated nalbuphine product is thought to be transported into the gastrointestinal tract via biliary secretion whereby the drug conjugate is cleaved liberating nalbuphine, which can be reabsorbed from the intestine. The sustained release formulation can improve the duration of anti-pruritic effect, by more slowly releasing nalbuphine into the in vivo system and allowing more drug to be conjugated and therefore available for recirculation and later reabsorption from the intestine.

The present methods can employ compositions including nalbuphine or a pharmaceutically acceptable salt, solvate or ester thereof and a sustained release delivery system. The sustained release delivery system includes (i) at least one hydrophilic compound, at least one cross-linking agent, and at least one pharmaceutical diluent; (ii) at least one hydrophilic compound, at least one cross-linking agent, at least one pharmaceutical diluent, and at least one cationic cross-linking agent different from the first cross-linking agent; or (iii) at least one hydrophilic compound, at least one cationic cross-linking compound, and at least one pharmaceutical diluent. Alternatively, in other embodiments, the present methods can employ compositions including nalbuphine or a pharmaceutically acceptable salt, solvate or ester thereof and a sustained release delivery system, which may employ a hydrophobic compound in a sustained release system.

The nalbuphine can be homogeneously dispersed in the sustained release delivery system. In some embodiments, the nalbuphine or pharmaceutically acceptable salt, solvate or ester thereof is present in the composition in an amount of about 1 mg to about 240 mg; about 1 mg to about 150 mg; about 1 mg to about 125 mg; or about 1 mg to about 100 mg. In some embodiments, the nalbuphine or pharmaceutically acceptable salt, solvate or ester thereof is present in the composition in an amount of about 5 mg to about 80 mg; about 10 mg to about 70 mg; about 15 mg to about 60 mg; about 40 mg to about 80 mg; about 50 mg to about 70 mg; or about 45 mg to about 60 mg. In one embodiment, the nalbuphine or pharmaceutically acceptable salt, solvate or ester thereof is present in the composition in an amount of about 15 mg, about 20 mg, about 25 mg, about 30 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, or about 240 mg. In another embodiment, the nalbuphine or pharmaceutically acceptable salt thereof is present in the composition in an amount of about 15 mg, about 30 mg, about 45 mg, about 60 mg, about 90 mg, about 120 mg, or about 180 mg.

In yet another embodiment, the nalbuphine or pharmaceutically acceptable salt thereof, e.g., HCL is present in the composition in an amount of about 15 mg, about 30 mg, about 60 mg, about 90 mg, about 120 mg, or about 180 mg.

In some embodiments, the sustained release delivery system is present in the composition in an amount from about 10 mg to about 420 mg; from about 25 mg to about 225 mg; from about 21 mg to about 198 mg; or from about 80 mg to about 200 mg; from about 80 mg to about 220 mg; from about 90 mg to about 210 mg; from about 100 mg to about 200 mg; from about 110 mg to about 190 mg; from about 120 mg to about 180 mg; from about 130 mg to about 170 mg; from about 140 mg to about 160 mg; from about 30 mg to about 60 mg; from about 60 mg to about 180 mg; from about 30 mg to about 180 mg, from about 75 mg to about 150 mg, from about 80 mg to about 160 mg, from about 90 mg to about 150 mg, from about 100 mg to about 140 mg, from about 110 mg to about 130 mg, from about 100 mg to about 300 mg, from about 200 mg to about 300 mg or from about 200 mg to about 250 mg. In one embodiment, the sustained release delivery system is present in the composition in an amount from about 75 mg to about 150 mg.

In some embodiments, the sustained release delivery system is present in the composition in an amount of about 30 mg, about 60 mg, about 75 mg, about 80 mg, about 90 mg, about 100 mg, about 110 mg, about 112 mg, about 115 mg, about 117 mg, about 120 mg, about 125 mg, about 130 mg, about 135 mg, about 140 mg, about 145 mg, about 150 mg, about 160 mg, about 170 mg, about 180 mg, about 190 mg, about 200 mg, about 210 mg, about 220 mg, about 225 mg, about 230 mg, about 240 mg, about 250 mg, about 260 mg, about 270 mg, about 280 mg, about 300 mg, about 320 mg, about 340 mg, about 360 mg, about 380 mg, about 400 mg or about 420 mg. In another embodiment, the sustained release delivery system is present in the composition in an amount of about 112 mg.

The ratio of nalbuphine or pharmaceutically acceptable salt, solvate or ester thereof in the compositions to the sustained release delivery system is generally from about 4:1 to about 1:25. In some embodiments, the ratio of nalbuphine or pharmaceutically acceptable salt, solvate or ester thereof to the sustained release delivery system is generally from about 2.5:1 to about 1:4. In some embodiments, the ratio of nalbuphine or pharmaceutically acceptable salt, solvate or ester thereof to the sustained release delivery system is generally from about 5:1 to about 1:5, about 4:1 to about 1:4, about 3:1 to about 1:3, about 2:1 to about 1:2, about 1:1 to about 1:5, about 1:1 to about 1:4, about 1:1 to about 1:3, about 1:1 to about 1.2, and about 1:2 to about 1:3. In some embodiments, the ratio of nalbuphine or pharmaceutically acceptable salt, solvate or ester thereof to the sustained release delivery system is about 1:1, about 1:2, about 1:2.5, about 1:3, about 1:4, or about 1:5.

In one embodiment, at least one hydrophilic compound is present in the sustained release delivery system in an amount of about 5% to about 80% by weight; the at least one cross-linking agent is present in the sustained release delivery system in an amount of about 0.5% to about 80% by weight; and the at least one pharmaceutical diluent is present in the sustained release delivery system in an amount of about 20% to about 80% by weight. In another embodiment, the at least one hydrophilic compound is present in the sustained release delivery system in an amount of about 8% to about 31% by weight; the at least one cross-linking agent is present in the sustained release delivery system in an amount of about 12% to about 47% by weight; and the at least one pharmaceutical diluent is present in the sustained release delivery system in an amount of about 20% to about 78% by weight. In another embodiment, the at least one hydrophilic compound is present in the sustained release delivery system in an amount of about 10% to about 20% by weight; the at least one cross-linking agent is present in the sustained release delivery system in an amount of about 15% to about 25% by weight; and the at least one pharmaceutical diluent is present in the sustained release delivery system in an amount of about 50% to about 85% by weight. In some embodiments, the at least one hydrophilic compound is present in the sustained release delivery system in an amount of about 8%, about 9%, about 10%, about 11%, about 12%, about 13%, about 14%, about 15%, about 16%, about 17%, about 18%, about 19%, about 20%, about 22%, about 24%, about 26%, about 28%, about 30%, about 32%, about 34%, or about 36% by weight; the at least one cross-linking agent is present in the sustained release delivery system in an amount of about 10%, about 11%, about 12%, about 13%, about 14%, about 15%, about 16%, about 17%, about 18%, about 19%, about 20%, about 22%, about 24%, about 26%, about 28%, about 30%, about 32%, about 33%, about 34%, or about 35% by weight; and the at least one pharmaceutical diluent is present in the sustained release delivery system in an amount of about 40%, about 45%, about 50%, about 55%, about 60%, about 65%, about 70%, about 80%, or about 85% by weight.

In some embodiments, the at least one hydrophilic compound is present in the sustained release delivery system in an amount of about 10%, about 11%, about 12%, about 13%, about 14%, about 15%, about 16%, about 17%, about 18%, about 19%, or about 20% by weight; the at least one cross-linking agent is present in the sustained release delivery system in an amount of about 15%, about 16%, about 17%, about 18%, about 19%, about 20%, or about 22% by weight; and the at least one pharmaceutical diluent is present in the sustained release delivery system in an amount of about 55%, about 60%, about 65%, about 70%, about 80%, or about 85% by weight. In one embodiment, the at least one hydrophilic compound is present in the sustained release delivery system in an amount of about 8%, about 12%, or about 20% by weight; the at least one cross-linking agent is present in the sustained release delivery system in an amount of about 12%, about 18%, or about 30% by weight; and the at least one pharmaceutical diluent is present in the sustained release delivery system in an amount of about 40%, about 60%, or about 70% by weight.

In one embodiment, nalbuphine is in the form of any pharmaceutically acceptable salt known in the art. Exemplary pharmaceutically acceptable salts include without limitation hydrochloric, sulfuric, nitric, phosphoric, hydrobromic, maleic, malic, ascorbic, citric, tartaric, pamoic, lauric, stearic, palmitic, oleic, myristic, lauryl sulfuric, napthalenesulfonic, linoleic, linolenic acid, and the like. One embodiment includes the hydrochloride salt of nalbuphine.

The sustained release delivery system includes at least one hydrophilic compound. The hydrophilic compound preferably forms a gel matrix that releases the nalbuphine or the pharmaceutically acceptable salt, solvate or ester thereof at a sustained rate upon exposure to liquids. The rate of release of the nalbuphine or the pharmaceutically acceptable salt, solvate or ester thereof from the gel matrix depends on the drug's partition coefficient between the components of the gel matrix and the aqueous phase within the gastrointestinal tract. The weight ratio of nalbuphine to hydrophilic compound is generally in the range of about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, and about 2:1 to about 1:2. In some embodiments, the weight ratio of nalbuphine to hydrophilic compound is in the range of about 10:1 to about 1:1, about 10:1 to about 2:1, about 9:1 to about 1:1, about 8:1 to about 1:1, about 7:1 to about 1:1, about 6:1 to about 1:1, about 5:1 to about 1:1, about 4:1 to about 1:1, about 3:1 to about 1:1, and about 2:1 to about 1:1. In some embodiments, the weight ratio of nalbuphine to hydrophilic compound is in the range of about 6:1 to about 1:1, about 5:1 to about 2:1, about 4:1 to about 3:1, about 4:1 to about 2:1, and about 5:1 to about 2:1. In some embodiments, the weight ratio of nalbuphine to hydrophilic compound is about 1:5, about 1:4.5, about 1:4.4, about 1:4, about 1:3.5, about 1:3.3, about 1:3, about 1:2.5, about 1:2, about 1:1, and about 1:1.5.

The sustained release delivery system generally includes the hydrophilic compound in an amount of about 5% to about 80% by weight. In some embodiments, the sustained release delivery system generally includes the hydrophilic compound in an amount of about 5% to about 30%, about 8% to about 31%, about 10% to about 20%, about 20% to about 60%, or about 40% to about 60% by weight. In one embodiment, the sustained release delivery system includes the hydrophilic compound in an amount of about 8% to about 31% by weight. In one embodiment, the sustained release delivery system includes the hydrophilic compound in an amount of about 10% to about 20% by weight. In some embodiments, the sustained release delivery system includes the hydrophilic compound in an amount of about 10%, about 11%, about 12%, about 13%, about 14%, about 15%, about 16%, about 17%, about 18%, about 19%, or about 20% by weight. In one embodiment, the sustained release delivery system includes the hydrophilic compound in an amount of about 12% by weight. In one embodiment, the sustained release delivery system includes the hydrophilic compound in an amount of about 8% by weight. In one embodiment, the sustained release delivery system includes the hydrophilic compound in an amount of about 20% by weight. In one embodiment, the sustained release delivery system includes the hydrophilic compound in an amount of about 28% by weight.

The hydrophilic compound is any pharmaceutically acceptable compound known in the art to be hydrophilic. Exemplary hydrophilic compounds include without limitation pharmaceutically acceptable gums, cellulose ethers, polyvinyl pyrrolidone, protein-derived compounds, and mixtures thereof. Exemplary gums include without limitation heteropolysaccharide gums and homopolysaccharide gums, such as xanthan, tragacanth, pectins, acacia, karaya, alginates, agar, guar, hydroxypropyl guar, carrageenan, locust bean gums, and gellan gums. Exemplary cellulose ethers include without limitation hydroxyalkyl celluloses and carboxyalkyl celluloses. In some embodiments, cellulose ethers include hydroxyethyl celluloses, hydroxypropyl celluloses, hydroxypropylmethyl-celluloses, carboxy methylcelluloses, and mixtures thereof. In some embodiments, the hydrophilic compound is a gum. In other embodiments, the hydrophilic compound is a heteropolysaccharide gum. In further embodiments, the hydrophilic compound is a xanthan gum or derivative thereof. Derivatives of xanthan gum include without limitation, for example, deacylated xanthan gum, the carboxymethyl esters of xanthan gum, and the propylene glycol esters of xanthan gum.

In another aspect, the sustained release delivery system further includes at least one cross-linking agent. In one embodiment, the cross-linking agent is a compound that is capable of cross-linking the hydrophilic compound to form a gel matrix in the presence of liquids. As used herein, “liquids” includes, for example, gastrointestinal fluids and aqueous solutions, such as those used for in vitro dissolution testing. The sustained release delivery system generally includes the cross-linking agent in an amount of about 0.5% to about 80% by weight. In one embodiment, the sustained release delivery system generally includes the cross-linking agent in an amount of about 12% to about 47% by weight. In another embodiment, the sustained release delivery system generally includes the cross-linking agent in an amount of about 20% to about 30% by weight. In one embodiment, the sustained release delivery system generally includes the cross-linking agent in an amount of about 15% to about 25% by weight. In some embodiments, the at least one cross-linking agent is present in the sustained release delivery system in an amount of about 15%, about 16%, about 17%, about 18%, about 19%, about 20%, about 21%, about 22%, about 23%, about 24%, or about 25% by weight. In one embodiment, the sustained release delivery system includes the cross-linking agent in an amount of about 18% by weight. In one embodiment, the sustained release delivery system includes the cross-linking agent in an amount of about 12% by weight. In one embodiment, the sustained release delivery system includes the cross-linking agent in an amount of about 30% by weight. In one embodiment, the sustained release delivery system includes the cross-linking agent in an amount of about 42% by weight.

Exemplary cross-linking agents include homopolysaccharides. Exemplary homopolysaccharides include without limitation galactomannan gums, such as guar gum, hydroxypropyl guar gum, and locust bean gum. In some embodiments, the cross-linking agent is a locust bean gum or a guar gum. In other embodiments, the cross-linking agent is an alginic acid derivative or hydrocolloid.

In some embodiments, when the sustained release delivery system includes at least one hydrophilic compound and at least one cross-linking agent, the weight ratio of hydrophilic compound to cross-linking agent is from about 1:9 to about 9:1, about 1:8 to about 8:1, about 1:7 to about 7:1, about 1:6 to about 6:1, about 1:5 to about 5:1, about 1:4 to about 4:1, about 1:3 to about 3:1, or about 1:2 to about 2:1. In some embodiments, the weight ratio of hydrophilic compound to cross-linking agent is about 1:5, about 1:4.5, about 1:4, about 1:3.5, about 1:3, about 1:2.5, about 1:2, about 1:1.5, and about 1:1.

When the sustained release delivery system includes at least one hydrophilic compound and at least one cross-linking agent, the weight ratio of the nalbuphine or pharmaceutically acceptable salt, solvate or ester thereof to the sum of the at least one hydrophilic compound and the at least one cross-linking agent is from about 10:1 to about 1:10, from about 9:1 to about 1:9, from about 8:1 to about 1:8, from about 7:1 to about 1:7, from about 6:1 to about 1:6, from about 5:1 to about 1:5, from about 4:1 to about 1:4, from about 3:1 to about 1:3, or from about 2:1 to about 1:2. In some embodiments, the weight ratio of the nalbuphine or pharmaceutically acceptable salt, solvate or ester thereof to the sum of the at least one hydrophilic compound and the at least one cross-linking agent is from about 4:1 to about 1:1, from about 4:1 to about 1:1.5, from about 3:1 to about 1:1, or from about 2:1 to about 1:1. In one embodiment, the ratio of the nalbuphine or pharmaceutically acceptable salt, solvate or ester thereof to the sum of the at least one hydrophilic compound and the at least one cross-linking agent is about 5:1, about 4:1 (i.e., 1:0.25), about 3.5:1, about 3:1, about 2.5:1, about 2:1 (i.e., 1:0.5), about 1.9:1, about 1.8:1, about 1.7:1, about 1.6:1, about 1.5:1, about 1.4:1, about 1.3:1, about 1.2:1, about 1.1:1, about 1:1, about 1:1.5, about 1:2, about 1:3, about 1:4, and about 1:5.

The sustained release delivery system further includes one or more pharmaceutical diluents known in the art. Exemplary pharmaceutical diluents include without limitation monosaccharides, disaccharides, polyhydric alcohols and mixtures thereof. In some embodiments, pharmaceutical diluents include, for example, starch, mannitol, lactose, dextrose, sucrose, microcrystalline cellulose, sorbitol, xylitol, fructose, and mixtures thereof. In some embodiments, the pharmaceutical diluent is water-soluble. Nonlimiting examples of water-soluble pharmaceutical diluents include lactose, dextrose, sucrose, or mixtures thereof. The weight ratio of pharmaceutical diluent to hydrophilic compound is generally from about 1:9 to about 9:1, from about 1:8 to about 8:1, from about 1:7 to about 7:1, from about 1:6 to about 6:1, from about 1:5 to about 5:1, from about 1:4 to about 4:1, from about 1:3 to about 3:1, or from about 1:2 to about 2:1. In some embodiments, the weight ratio of pharmaceutical diluent to hydrophilic compound is generally from about 9:1 to about 1:1.5. In some embodiments, the weight ratio of pharmaceutical diluent to hydrophilic compound is about 9:1, about 8.75:1, about 8.5:1, about 8.25:1, about 8:1, about 7.5:1, about 7:1, about 6.5:1, about 6:1, about 5.5:1, about 5:1, about 4.5:1, about 4:1, about 3.5:1, about 3:1, about 2.5:1, about 2:1, about 1.5:1, or about 1:1.

The sustained release delivery system generally includes one or more pharmaceutical diluents in an amount of about 20% to about 80%, about 30% to about 70%, about 40% to about 70%, or about 40% to about 60%. In one embodiment, the sustained release delivery system includes one or more pharmaceutical diluents in an amount of about 20% to about 70% by weight. In one embodiment, the sustained release delivery system includes one or more pharmaceutical diluents in an amount of about 50% to about 85% by weight. In some embodiments, the sustained release delivery system includes one or more pharmaceutical diluents in an amount of about 55%, about 60%, about 65%, about 70%, about 80%, or about 85% by weight. In one embodiment, the sustained release delivery system includes one or more pharmaceutical diluents in an amount of about 20% by weight. In one embodiment, the sustained release delivery system includes one or more pharmaceutical diluents in an amount of about 30% by weight. In one embodiment, the sustained release delivery system includes one or more pharmaceutical diluents in an amount of about 40% by weight. In one embodiment, the sustained release delivery system includes one or more pharmaceutical diluents in an amount of about 50% by weight. In one embodiment, the sustained release delivery system includes one or more pharmaceutical diluents in an amount of about 60% by weight. In one embodiment, the sustained release delivery system includes one or more pharmaceutical diluents in an amount of about 70% by weight.

In a further aspect, the sustained release delivery system includes one or more cationic cross-linking compounds. In some embodiments, the one or more cationic cross-linking compounds are used instead of the cross-linking agent. In some embodiments, the one or more cationic cross-linking compounds are used in addition to the cross-linking agent. In one embodiment, the one or more cationic cross-linking compounds are used in an amount sufficient to cross-link the hydrophilic compound to form a gel matrix in the presence of liquids. In some embodiments, the one or more cationic cross-linking compounds are present in the sustained release delivery system in an amount of about 0.5% to about 30%, about 0.5% to about 25%, about 0.5% to about 20%, about 0.5% to about 15%, about 0.5% to about 10%, or about 0.5% to about 5% by weight. In some embodiments, the one or more cationic cross-linking compounds are present in the sustained release delivery system in an amount of about 5% to about 20%, about 5% to about 15%, about 6% to about 14%, about 7% to about 13%, about 8% to about 12%, or about 9% to about 11% by weight. In some embodiments, the one or more cationic cross-linking compounds are present in the sustained release delivery system in an amount of about 5%, about 6%, about 7%, about 8%, about 9%, about 10%, about 11%, about 12%, about 13%, about 14%, or about 15% by weight. In one embodiment, the cationic cross-linking compound is present in the sustained release delivery system in an amount of about 10% by weight.

Exemplary cationic cross-linking compounds include without limitation monovalent metal cations, multivalent metal cations, and inorganic salts, including alkali metal and/or alkaline earth metal sulfates, chlorides, borates, bromides, citrates, acetates, lactates, and mixtures thereof. For example, the cationic cross-linking compound include without limitation one or more of calcium sulfate, sodium chloride, potassium sulfate, sodium carbonate, lithium chloride, tripotassium phosphate, sodium borate, potassium bromide, potassium fluoride, sodium bicarbonate, calcium chloride, magnesium chloride, sodium citrate, sodium acetate, calcium lactate, magnesium sulfate, sodium fluoride, or mixtures thereof.

When the sustained release delivery system includes at least one hydrophilic compound and at least one cationic cross-linking compound, the weight ratio of hydrophilic compound to cationic cross-linking compound ranges from about 1:9 to about 9:1, from about 1:8 to about 8:1, from about 1:7 to about 7:1, from about 1:6 to about 6:1, from about 1:5 to about 5:1, from about 1:4 to about 4:1, from about 1:3 to about 3:1, or from about 1:2 to about 2:1. In one embodiment, the weight ratio of hydrophilic compound to cationic cross-linking compound ranges from about 1:3 to about 3:1. In some embodiments, the weight ratio of hydrophilic compound to cationic cross-linking compound is about 3:1, about 2.75:1, about 2.5:1, about 2.25:1, about 2:1, about 1.8:1, about 1.6:1, about 1.4:1, about 1.2:1, about 1:1, about 1:1.25, about 1:1.5, or about 1:2. In one embodiment, the weight ratio of hydrophilic compound to cationic cross-linking compound is about 1:1.25. In one embodiment, the weight ratio of hydrophilic compound to cationic cross-linking compound is about 1.2:1. In one embodiment, the weight ratio of hydrophilic compound to cationic cross-linking compound is about 2:1. In one embodiment, the weight ratio of hydrophilic compound to cationic cross-linking compound is about 2.8:1.

In one embodiment, the at least one hydrophilic compound is present in the sustained release delivery system in an amount of about 5% to about 80% by weight; the at least one cationic cross-linking agent is present in the sustained release delivery system in an amount of about 0.5% to about 30% by weight; and the at least one pharmaceutical diluent is present in the sustained release delivery system in an amount of about 20% to about 80% by weight. In another embodiment, the at least one hydrophilic compound is present in the sustained release delivery system in an amount of about 8% to about 30% by weight; the at least one cationic cross-linking agent is present in the sustained release delivery system in an amount of about 10% by weight; and the at least one pharmaceutical diluent is present in the sustained release delivery system in an amount of about 20% to about 70% by weight. In another embodiment, the at least one hydrophilic compound is present in the sustained release delivery system in an amount of about 5% to about 30% by weight; the at least one cationic cross-linking agent is present in the sustained release delivery system in an amount of about 5% to about 20% by weight; and the at least one pharmaceutical diluent is present in the sustained release delivery system in an amount of about 20% to about 85% by weight. In another embodiment, the at least one hydrophilic compound is present in the sustained release delivery system in an amount of about 10% to about 20% by weight; the at least one cationic cross-linking agent is present in the sustained release delivery system in an amount of about 5% to about 15% by weight; and the at least one pharmaceutical diluent is present in the sustained release delivery system in an amount of about 50% to about 85% by weight.

In some embodiments, the at least one hydrophilic compound is present in the sustained release delivery system in an amount of about 8%, about 9%, about 10%, about 11%, about 12%, about 13%, about 14%, about 15%, about 16%, about 17%, about 18%, about 19%, about 20%, about 22%, about 24%, about 26%, about 28%, or about 30% by weight; the at least one cationic cross-linking agent is present in the sustained release delivery system in an amount of about 5%, about 6%, about 7%, about 8%, about 9%, about 10%, about 11%, about 12%, about 13%, about 14%, about 15%, about 16%, about 17%, about 18%, about 19%, or about 20%, by weight; and the at least one pharmaceutical diluent is present in the sustained release delivery system in an amount of about 40%, about 45%, about 50%, about 55%, about 60%, about 65%, about 70%, about 80%, or about 85% by weight. In one embodiment, the at least one hydrophilic compound is present in the sustained release delivery system in an amount of about 10%, about 11%, about 12%, about 13%, about 14%, about 15%, about 16%, about 17%, about 18%, about 19%, or about 20% by weight; the at least one cationic cross-linking agent is present in the sustained release delivery system in an amount of about 5%, about 6%, about 7%, about 8%, about 9%, about 10%, about 11%, about 12%, about 13%, about 14%, about 15%, by weight; and the at least one pharmaceutical diluent is present in the sustained release delivery system in an amount of about 55%, about 60%, about 65%, about 70%, about 80%, or about 85% by weight. In one embodiment, the at least one hydrophilic compound is present in the sustained release delivery system in an amount of about 8%, about 12%, or about 20% by weight; the at least one cationic cross-linking agent is present in the sustained release delivery system in an amount of about 10%, about 12%, or about 14% by weight; and the at least one pharmaceutical diluent is present in the sustained release delivery system in an amount of about 40%, about 60%, or about 70% by weight.

In one embodiment, the sustained release delivery system includes about 0.5% to about 80% locust bean gum, about 5% to about 80% xanthan gum, about 20% to about 80% mannitol and about 0.5% to 80% calcium sulfate dihydrate. In one embodiment, the sustained release delivery system includes about 12% to about 47% locust bean gum, about 8% to about 31% xanthan gum, about 20% to about 78% mannitol and about 0.5% to 25% calcium sulfate dihydrate. In one embodiment, the sustained release delivery system includes about 15% to about 25% locust bean gum, about 10% to about 20% xanthan gum, about 50% to about 85% mannitol and about 5% to 15% calcium sulfate dihydrate. In one embodiment, the sustained release delivery system includes about 18% locust bean gum, about 12% xanthan gum, about 60% mannitol and about 10% calcium sulfate dihydrate. In one embodiment, the sustained release delivery system includes about 12% locust bean gum, about 8% xanthan gum, about 70% mannitol and about 10% calcium sulfate dihydrate. In one embodiment, the sustained release delivery system includes about 20% locust bean gum, about 30% xanthan gum, about 40% mannitol and about 10% calcium sulfate dihydrate. In one embodiment, the sustained release delivery system includes about 30% locust bean gum, about 20% xanthan gum, about 40% mannitol and about 10% calcium sulfate dihydrate. In one embodiment, the sustained release delivery system includes about 42% locust bean gum, about 28% xanthan gum, about 20% mannitol and about 10% calcium sulfate dihydrate.

Two properties of the components of this sustained release system (e.g., the at least one hydrophilic compound and the at least one cross-linking agent; or the at least one hydrophilic compound and at least one cationic cross-linking compound) are that it forms a gel matrix upon exposure to liquids are fast hydration of the compounds/agents and the ability to form a gel matrix having a high gel strength. These two properties, which are needed to achieve a slow release gel matrix, are maximized by the particular combination of compounds (e.g., the at least one hydrophilic compound and the at least one cross-linking agent; or the at least one hydrophilic compound and the at least one cationic cross-linking compound). For example, hydrophilic compounds (e.g., xanthan gum) have excellent water-wicking properties that provide fast hydration. The combination of hydrophilic compounds with materials that are capable of cross-linking the rigid helical ordered structure of the hydrophilic compound (e.g., cross-linking agents and/or cationic cross-linking compounds) thereby acts synergistically to provide a higher than expected viscosity (i.e., high gel strength) of the gel matrix.

In some embodiments, the sustained release compositions are further admixed with one or more wetting agents (e.g., polyethoxylated castor oil, polyethoxylated hydrogenated castor oil, polyethoxylated fatty acid from castor oil, polyethoxylated fatty acid from hydrogenated castor oil) one or more lubricants (e.g., magnesium stearate, sodium stearyl fumarate, and the like), one or more buffering agents, one or more colorants, and/or other conventional ingredients.

In some embodiments, compositions employed in the present methods can contain additional pharmaceutical excipients. For example, in certain embodiments, fumaric acid can be added to the formulations described herein.

In other embodiments, a non-functional coating, e.g., Opadry® can be added to the compositions described herein.

In some embodiments, the compositions described herein further include a second hydrophilic compound. In some embodiments, the second hydrophilic compound is a cellulose ether. In some embodiments, the second hydrophilic compound is a hydroxyalkyl cellulose or a carboxyalkyl cellulose. In some embodiments, the second hydrophilic compound is a hydroxyethyl cellulose, a hydroxypropyl cellulose, a hydroxypropylmethyl-cellulose, a carboxy methylcellulose, or a mixture thereof. In some embodiments, the second hydrophilic is an ethyl cellulose or wax (e.g., including without limitation cetyl alcohol, stearyl alcohol, white wax, or carnauba wax). The second hydrophilic compound is present in the formulation in an amount ranging from about 5% to about 45%, about 5% to about 25%, about 10% to about 20%, or 12% to about 18% by weight. In some embodiments, the second hydrophilic compound is present in the formulation in an amount of about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 30%, about 35%, about 40%, or about 45%.

In some embodiments, the weight ratio of the second hydrophilic compound to the nalbuphine or pharmaceutically acceptable salt, solvate or ester ranges from about 5:1 to about 1:5, about 4:1 to about 1:4, about 3:1 to about 1:3, about 2:1 to about 1:2, about 1:1 to about 1:3, or about 1:1 to about 1:2. In some embodiments, the weight ratio of the second hydrophilic compound to the nalbuphine or pharmaceutically acceptable salt, solvate or ester is about 5:1, about 4:1, about 3:1, about 2:1, about 1:1, about 1:2, about 1:3, about 1:4, or about 1:5.

In some embodiments, the weight ratio of the second hydrophilic compound to the sustained release delivery system ranges from about 10:1 to about 1:10, about 8:1 to about 1:8, about 6:1 to about 1:6, about 4:1 to about 1:4, about 2:1 to about 1:3, about 1:1 to about 1:10, about 1:1 to about 1:6, or about 1:2 to about 1:6. In some embodiments, the weight ratio of the second hydrophilic compound to the sustained release delivery system is about 10:1, about 8:1, about 6:1, about 4:1, about 2:1, about 1:1, about 1:1.5, about 1:2, about 1:2.5, about 1:3, about 1:4, about 1:5, about 1:6, about 1:7, about 1:8, about 1:9 or about 1:10.

In some embodiments, the oral sustained release solid dosage formulations including from about 1 mg to 200 mg nalbuphine hydrochloride and about 10 mg to about 420 mg of a sustained release delivery system. In these embodiments, the sustained release delivery system includes about 12% to about 42% locust bean gum; about 8.0% to about 28% xanthan gum; about 20% to about 70% mannitol; and about 5% to about 20% calcium sulfate dihydrate. In some embodiments, the present methods can employ oral sustained release solid dosage formulations including from about 5 mg to about 80 mg nalbuphine hydrochloride and about 80 mg to about 360 mg of a sustained release delivery system. In some embodiments, the present methods can employ oral sustained release solid dosage formulations including from about 50 mg to about 150 mg nalbuphine hydrochloride and about 100 mg to about 300 mg of a sustained release delivery system.

In some embodiments, the present methods employ oral sustained release solid dosage formulations including about 15 mg nalbuphine hydrochloride, and from about 25 mg to about 225 mg, for example about 195 mg, of a sustained release delivery system. In these embodiments, the sustained release delivery system includes about 14% locust bean gum; about 9% xanthan gum; about 47% mannitol; and about 8% calcium sulfate dihydrate.

In some embodiments, the present methods employ oral sustained release solid dosage formulations including about 30 mg nalbuphine hydrochloride, and from about 25 mg to about 225 mg, for example about 180 mg, of a sustained release delivery system. In these embodiments, the sustained release delivery system includes about 18% locust bean gum; about 12% xanthan gum; about 60% mannitol; and about 10% calcium sulfate dihydrate.

In some embodiments, the present methods employ oral sustained release solid dosage formulations including about 60 mg nalbuphine hydrochloride, and from about 25 mg to about 225 mg, for example about 120 mg, of a sustained release delivery system. In these embodiments, the sustained release delivery system includes about 10% locust bean gum; about 12% xanthan gum; about 60% mannitol; and about 10% calcium sulfate dihydrate. In some embodiments, the present methods employ oral sustained release solid dosage formulations including from about 5 mg to about 80 mg nalbuphine hydrochloride and about 80 mg to about 360 mg of a sustained release delivery system.

In some embodiments, the present methods employ oral sustained release solid dosage formulations including about 120 mg nalbuphine hydrochloride, and from about 25 mg to about 250 mg, for example about 240 mg, of a sustained release delivery system. In these embodiments, the sustained release delivery system includes about 18% locust bean gum; about 12% xanthan gum; about 60% mannitol; and about 10% calcium sulfate dihydrate.

In some embodiments, the present methods employ oral sustained release solid dosage formulations including about 30 mg nalbuphine hydrochloride, and from about 25 mg to about 350 mg, for example about 270 mg or about 360 mg, of a sustained release delivery system. In these embodiments, the sustained release delivery system includes about 18% locust bean gum; about 12% xanthan gum; about 60% mannitol; and about 10% calcium sulfate dihydrate.

In some embodiments, the present methods employ oral sustained release solid dosage formulations including about 45 to about 60 mg nalbuphine hydrochloride and from about 100 mg to about 200 mg of a sustained release delivery system. In these embodiments, the sustained release delivery system includes about 15% to about 25% locust bean gum; about 10% to about 20% xanthan gum; about 50% to about 85% mannitol; and about 5% to about 15% calcium sulfate dihydrate.

In some embodiments, the present methods employ oral sustained release solid dosage formulations including about 30 mg nalbuphine hydrochloride, about 32.4 mg locust bean gum; about 21.6 mg xanthan gum; about 108 mg mannitol; about 18 mg calcium sulfate dihydrate, about 35 mg hydroxypropylcellulose, and about 1.9 mg magnesium stearate.

In some embodiments, the present methods employ oral sustained release solid dosage formulations including about 60 mg nalbuphine hydrochloride, about 21.6 mg locust bean gum; about 14.4 mg xanthan gum; about 72 mg mannitol; about 12 mg calcium sulfate dihydrate, about 30 mg hydroxypropylcellulose, and about 1.6 mg magnesium stearate.

In some embodiments, the present methods employ oral sustained release solid dosage formulations including about 120 mg nalbuphine hydrochloride, about 43.2 mg locust bean gum; about 28.8 mg xanthan gum; about 144 mg mannitol; about 24 mg calcium sulfate dihydrate, about 60 mg hydroxypropylcellulose, and about 3.2 mg magnesium stearate.

In some embodiments, the present methods employ oral sustained release solid dosage formulations including about 180 mg nalbuphine hydrochloride, about 64.8 mg locust bean gum; about 43.2 mg xanthan gum; about 216 mg mannitol; about 36 mg calcium sulfate dihydrate, about 90 mg hydroxypropylcellulose, about 5 mg magnesium stearate, and about 25 mg fumaric acid.

In some embodiments, the present methods employ oral sustained release solid dosage formulations including about 180 mg nalbuphine hydrochloride, about 48.6 mg locust bean gum; about 32.4 mg xanthan gum; about 162 mg mannitol; about 27 mg calcium sulfate dihydrate, about 60 mg hydroxypropylcellulose, about 4 mg magnesium stearate, and about 25 mg fumaric acid.

In some embodiments, the present methods employ oral sustained release solid dosage formulations including about 30 mg nalbuphine hydrochloride, about 32.4 mg locust bean gum; about 21.6 mg xanthan gum; about 108 mg mannitol; about 18 mg calcium sulfate dihydrate, about 35 mg hydroxypropylcellulose, about 1.9 mg magnesium stearate, and about 7.4 mg Opadry II White.

The sustained release formulations of nalbuphine are orally administrable solid dosage formulations. Nonlimiting examples of oral solid dosage formulations include tablets, capsules including a plurality of granules, sublingual tablets, powders, granules, syrups, and buccal dosage forms or devices (e.g., buccal patches, tablets, etc.). In some embodiments, tablets have an enteric coating or a hydrophilic coating.

The sustained release delivery system is prepared by dry granulation or wet granulation, before the nalbuphine or pharmaceutically acceptable salt, solvate or ester thereof is added, although the components can be held together by an agglomeration technique to produce an acceptable product. In the wet granulation technique, the components (e.g., hydrophilic compounds, cross-linking agents, pharmaceutical diluents, cationic cross-linking compounds, hydrophobic polymers, etc.) are mixed together and then moistened with one or more liquids (e.g., water, propylene glycol, glycerol, alcohol) to produce a moistened mass that is subsequently dried. The dried mass is then milled with conventional equipment into granules of the sustained release delivery system. Thereafter, the sustained release delivery system is mixed in the desired amounts with the nalbuphine or the pharmaceutically acceptable salt, solvate or ester thereof and, optionally, one or more wetting agents, one or more lubricants, one or more buffering agents, one or more coloring agents, one or more second hydrophilic compounds, or other conventional ingredients, to produce a granulated composition. The sustained release delivery system and the nalbuphine can be blended with, for example, a high shear mixer. The nalbuphine is preferably finely and homogeneously dispersed in the sustained release delivery system. The granulated composition, in an amount sufficient to make a uniform batch of tablets, is subjected to tableting in a conventional production scale tableting machine at typical compression pressures, i.e., about 2,000-16,000 psi. In some embodiments, the mixture should not be compressed to a point where there is subsequent difficulty with hydration upon exposure to liquids.

In some embodiments, the nalbuphine formulation is prepared by dry granulation or wet granulation. The components of the sustained release delivery system are added, along with the nalbuphine or a pharmaceutically acceptable salt, solvate or ester thereof. Alternatively, all of the components can be held together by an agglomeration technique to produce an acceptable product. In the wet granulation technique, nalbuphine or pharmaceutically salt, solvate or ester thereof and the components (e.g., hydrophilic compounds, cross-linking agents, pharmaceutical diluents, cationic cross-linking compounds, hydrophobic polymers, etc.) are mixed together and then moistened with one or more liquids (e.g., water, propylene glycol, glycerol, alcohol) to produce a moistened mass that is subsequently dried. The dried mass is then milled with conventional equipment into granules. Optionally, one or more wetting agents, one or more lubricants, one or more buffering agents, one or more coloring agents, one or more second hydrophilic compounds, or other conventional ingredients, are also added to the granulation. The granulated composition, in an amount sufficient to make a uniform batch of tablets, is subjected to tableting in a conventional production scale tableting machine at typical compression pressures, i.e., about 2,000-16,000 psi. In some embodiments, the mixture should not be compressed to a point where there is subsequent difficulty with hydration upon exposure to liquids.

The average particle size of the granulated composition is from about 50 m to about 400 m by weight. In some embodiments, the average particle size by weight is from about 185 m to about 265 m. The average density of the granulated composition is from about 0.3 g/mL to about 0.8 g/mL. In some embodiments, the average density is from about 0.5 g/mL to about 0.7 g/mL. The tablets formed from the granulations are generally from about 4 Kp to about 22 Kp hardness. The average flow of the granulations is from about 25 to about 40 g/sec.

In some embodiments, the present methods can employ a multilayer solid dosage form, in which the layers are formulated to release the nalbuphine hydrochloride at different rates. For example, in one embodiment, the second layer is an extended release layer that includes nalbuphine or a pharmaceutically acceptable salt, solvate or ester thereof and a sustained release delivery system designed to release the nalbuphine or the pharmaceutically acceptable salt, solvate or ester thereof at a controlled rate so that therapeutically effective blood levels are maintained over an extended period of time (e.g., from about 8 to about 12 hours). The first layer is an immediate release layer that includes a formulation of nalbuphine or a pharmaceutically acceptable salt, solvate or ester thereof designed to release the nalbuphine or the pharmaceutically acceptable salt, solvate or ester thereof at a rate that is faster than the rate of the second layer to achieve a therapeutically effective blood level in an immediate period of time (e.g., from about 1 to about 2 hours). In some embodiments, the first layer includes a sustained release delivery system. In some embodiments, the first layer does not include a sustained release delivery system.

In some embodiments, the weight ratio of the second layer to the first layer is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, about 2:1 to about 1:2. In one embodiment, the weight ratio of the second layer to the first layer is about 5:1 to about 1:5. In a further embodiment, the weight ratio of the second layer to the first layer is about 1:1 to about 1:2. In some embodiments, the weight ratio of the second layer to the first layer is about 1:1, about 1:1.2, about 1:1.4, about 1:1.6, about 1:1.8, or about 1:2. In one embodiment, the weight ratio of the second layer to the first layer is about 1:2. In one embodiment, the weight ratio of the second layer to the first layer is about 1:1.4. In some embodiments, the weight ratio of the second layer to the first layer is about 3:1, about 2.5:1, about 2:1, about 1.5:1. In one embodiment, the weight ratio of the second layer to the first layer is about 2.5:1.

The sustained release delivery system of the multilayer dosage form includes (i) at least one hydrophilic compound, at least one cross-linking agent, and at least one pharmaceutical diluent; (ii) at least one hydrophilic compound, at least one cross-linking agent, at least one pharmaceutical diluent, and at least one cationic cross-linking agent different from the first cross-linking agent; or (iii) at least one hydrophilic compound, at least one cationic cross-linking compound, and at least one pharmaceutical diluent. In some embodiments, when the first layer includes a sustained release delivery system, the sustained release delivery system of the first layer includes the same components as the sustained release delivery system of the second layer (e.g., both the first and second layers are one of embodiments (i)-(iii), listed above). In other embodiments, the sustained release delivery system of the first layer includes different components as the sustained release delivery system of the second layer (e.g., the first layer is embodiment (i), listed above, while the second layer is embodiment (iii), listed above). It is recognized that the sustained release delivery system of either layer can be one of embodiments (i)-(iii) listed above. Moreover, it is recognized that in some embodiments, the first layer does not include a sustained release delivery system.

The sustained release delivery system is generally present in the second layer (e.g., extended release layer) in an amount ranging from about 10 mg to about 420 mg. In some embodiments, the sustained release delivery system is present in the second layer in an amount ranging from about 110 mg to about 200 mg. In some embodiments, the sustained release delivery system is present in the second layer in an amount ranging from about 110 mg to about 150 mg. In some embodiments, the sustained release delivery system is present in the second layer in an amount ranging from about 90 mg to about 150 mg. In some embodiments, the sustained release delivery system is present in the second layer in an amount of about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg. In one embodiment, the sustained release delivery system is present in the second layer in an amount of about 123 mg. In one embodiment, the sustained release delivery system is present in the second layer in an amount of about 101 mg. In one embodiment, the sustained release delivery system is present in the second layer in an amount of about 92 mg. In another embodiment, the sustained release delivery system is present in the second layer in an amount of about 112.5 mg. In one embodiment, the sustained release delivery system is present in the second layer in an amount of about 135 mg. In one embodiment, the sustained release delivery system is present in the second layer in an amount of about 150 mg.

Nalbuphine or a pharmaceutically acceptable salt, solvate or ester thereof is generally present in the second layer in an amount ranging from about 15 mg to about 60 mg. In some embodiments, nalbuphine or a pharmaceutically acceptable salt, solvate or ester thereof is present in the second layer in an amount ranging from about 30 mg to about 60 mg. In some embodiments, nalbuphine or a pharmaceutically acceptable salt, solvate or ester thereof is present in the second layer in an amount ranging from about 45 mg to about 60 mg. In one embodiment, nalbuphine or a pharmaceutically acceptable salt, solvate or ester thereof is present in the second layer in an amount of about 15 mg. In one embodiment, nalbuphine or a pharmaceutically acceptable salt, solvate or ester thereof is present in the second layer in an amount of about 30 mg. In one embodiment, nalbuphine or a pharmaceutically acceptable salt, solvate or ester thereof is present in the second layer in an amount of about 45 mg. In one embodiment, nalbuphine or a pharmaceutically acceptable salt, solvate or ester thereof is present in the second layer in an amount of about 15 mg, about 30 mg, about 60 mg, about 90 mg, about 120 mg, or about 180 mg.

In some embodiments, the weight ratio of nalbuphine or a pharmaceutically acceptable salt, solvate or ester thereof to the sustained release delivery system in the second layer is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, or about 2:1 to about 1:2. In one embodiment, the weight ratio of nalbuphine or a pharmaceutically acceptable salt, solvate or ester thereof to the sustained release delivery system in the second layer is about 1:2 to about 1:4. In one embodiment, the weight ratio of nalbuphine or a pharmaceutically acceptable salt, solvate or ester thereof to the sustained release delivery system in the second layer is about 1:1 to about 1:5. In some embodiments, the weight ratio of nalbuphine or a pharmaceutically acceptable salt, solvate or ester thereof to the sustained release delivery system in the second layer is about 1:1, about 1:1.2, about 1:1.4, about 1:1.6, about 1:1.8, about 1:2, about 1:2.5, about 1:3, or about 1:3.5. In one embodiment, the weight ratio of nalbuphine or a pharmaceutically acceptable salt, solvate or ester thereof to the sustained release delivery system in the second layer is about 1:2.5. In another embodiment, the weight ratio of nalbuphine or a pharmaceutically acceptable salt, solvate or ester thereof to the sustained release delivery system in the second layer is about 1:3.3. In a further embodiment, the weight ratio of nalbuphine or a pharmaceutically acceptable salt, solvate or ester thereof to the sustained release delivery system in the second layer is about 1:3. In yet another embodiment, the ratio of nalbuphine or a pharmaceutically acceptable salt, solvate or ester thereof to the sustained release delivery system in the second layer is about 1:2.

When the sustained release delivery system is present in the first layer (e.g., immediate release layer), it is generally present in an amount ranging from about 0 mg to about 50 mg. In some embodiments, the sustained release delivery system is present in the first layer in an amount ranging from about 5 mg to about 25 mg or from about 5 mg to about 15 mg. In one embodiment, the sustained release delivery system is present in the first layer in an amount of about 3 mg to about 9 mg. In one embodiment, the sustained release delivery system is present in the first layer in an amount of about 4 mg to about 6 mg. In some embodiments, the sustained release delivery system is present in the first layer in an amount of about 2 mg, about 4 mg, about 6 mg, about 8 mg, about 10 mg, about 12 mg, about 14 mg, about 15 mg, about 16 mg, about 18 mg, about 20 mg about 25 mg, about 30 mg, about 35 mg, about 40 mg, about 45 mg or about 50 mg. In one embodiment, the sustained release delivery system is present in the first layer in an amount of about 6 mg.

In some embodiments, nalbuphine or a pharmaceutically acceptable salt, solvate or ester thereof is generally present in the first layer (e.g., immediate release layer) in an amount ranging from about 5 mg to about 180 mg. In some embodiments, nalbuphine or a pharmaceutically acceptable salt, solvate or ester thereof is present in the first layer in an amount ranging from about 5 mg to about 25 mg or from about 10 mg to about 20 mg. In some embodiments, the nalbuphine or a pharmaceutically acceptable salt, solvate or ester thereof is present in the first layer in an amount of about 5 mg, about 10 mg, about 11 mg, about 12 mg, about 13 mg, about 14 mg, about 15 mg, about 16 mg, about 17 mg, about 18 mg, about 19 mg, about 20 mg, about 25 mg, about 30 mg, about 35 mg, about 40 mg, about 45 mg or about 50 mg. In one embodiment, nalbuphine or a pharmaceutically acceptable salt, solvate or ester thereof is present in the first layer in an amount of about 15 mg, about 30 mg, about 60 mg, about 90 mg, about 120 mg, or about 180 mg.

In some embodiments, when the first layer includes a sustained release delivery system, the ratio of nalbuphine or pharmaceutically acceptable salt, solvate or ester thereof to the sustained release delivery system in the first layer is about 10:1 to about 1:10, about 9:1 to about 1:9, about 8:1 to about 1:8, about 7:1 to about 1:7, about 6:1 to about 1:6, about 5:1 to about 1:5, about 4:1 to about 1:4, about 3:1 to about 1:3, about 2:1 to about 1:2. In one embodiment, the ratio of nalbuphine or pharmaceutically acceptable salt, solvate or ester thereof to the sustained release delivery system in the first layer is about 2:1 to about 4:1. In some embodiments, the ratio of nalbuphine or pharmaceutically acceptable salt, solvate or ester thereof to the sustained release delivery system in the first layer is about 5:1, about 4.5:1, about 4:1, about 3.5:1, about 3:1, about 2.5:1, about 2:1, about 1.5:1, or about 1:1. In one embodiment, the ratio of nalbuphine or pharmaceutically acceptable salt, solvate or ester thereof to the sustained release delivery system in the first layer is about 2.5:1. In another embodiment, the ratio of nalbuphine or pharmaceutically acceptable salt, solvate or ester thereof to the sustained release delivery system in the first layer is about 3:1.

In some embodiments, the multilayer dosage form further includes a pharmaceutical disintegrant. The disintegrant promotes the dissolution and absorption of nalbuphine or pharmaceutically acceptable salt, solvate or ester thereof from the immediate release layer. Nonlimiting examples of pharmaceutical disintegrants include croscarmellose sodium, starch glycolate, crospovidone, and unmodified starch. In one embodiment, the disintegrant is in the first layer (i.e., the immediate release layer), of the dosage form. The disintegrant is generally present in the layer in an amount of about 1.5 mg to about 4.5 mg. In one embodiment, the disintegrant is present in an amount of about 3 mg. In one embodiment, the disintegrant is present in the layer in an amount of about 2-10% by weight. In one embodiment, the disintegrant is present in the layer in an amount of about 5% by weight. When the layer contains a sustained release delivery system, the weight ratio of the sustained release delivery system to the disintegrant is in a range of about 5:1 to about 1:5. In some embodiments, the ratio of the sustained release delivery system to the disintegrant is in a range of about 1:1 to about 3:1. In other embodiments, the ratio of the sustained release delivery system to the disintegrant is in a range of about 2:1.

In some embodiments, the multilayer tablets are prepared by first preparing the immediate release layer and extended release layer blends separately. The extended release layer is prepared as described above. The wet granulation of the extended release layer is then dried and milled to an appropriate size. Magnesium stearate is added and mixed with the milled granulation. The immediate release layer is prepared by first mixing the nalbuphine or the pharmaceutically acceptable salt, solvate or ester thereof with one or more diluents (e.g., microcrystalline cellulose). This mix is then optionally mixed with one or more disintegrants. The blend is mixed with magnesium stearate. Finally, the immediate release layer blend and the extended release layer blend are compressed into multi-layer (e.g., bi-layer) tablets.

In certain embodiments, the chemistry of certain of the components of the formulation, such as the hydrophilic compound (e.g., xanthan gum), is such that the components are considered to be self-buffering agents which are substantially insensitive to the solubility of the nalbuphine and the pH changes along the length of the gastrointestinal tract. Moreover, the chemistry of the components is believed to be similar to certain known muco-adhesive substances, such as polycarbophil. Muco-adhesive properties are desirable for buccal delivery systems. Thus, the sustained release formulation can loosely interact with the mucin in the gastrointestinal tract and thereby provide another mode by which a constant rate of delivery of the nalbuphine is achieved.

The phenomenon discussed above (muco-adhesive properties) is a mechanism by which the sustained release formulations can interact with the mucin and fluids of the gastrointestinal tract and provide a constant rate of delivery of the nalbuphine.

When measured by USP Procedure Drug Release General Chapter <711> Dissolution, (incorporated by reference herein in its entirety), the sustained release formulations employed in the present methods generally exhibit an in vitro dissolution of about 15% to about 50% by weight nalbuphine after 1 hour, about 45% to about 80% by weight nalbuphine after 4 hours, or at least about 80% by weight nalbuphine after 10 hours. In some embodiments, the in vitro and in vivo release characteristics of the sustained release formulations are modified using mixtures of one or more different water insoluble and/or water soluble compounds, using different plasticizers, varying the thickness of the sustained release film, including providing release-modifying compounds in the coating, and/or by providing passageways through the coating. In some embodiments, the dissolution rate is determined using apparatus USP Type III/250 mL at pH 6.8, 37° C. and 15 dpm. In some embodiments, the dissolution rate is determined using apparatus USP Type III/250 mL performed in pH change (0-1 hours pH 1.2, after hour 1 pH 4.5, after hour 2 pH 6.8) at 37° C. and 15 dpm.

In some embodiments, the sustained release formulation has an in vitro dissolution of about 50% to about 100% by weight nalbuphine after about 6 hours. In some embodiments, the sustained release formulation has an in vitro dissolution of about 75% to about 100% by weight nalbuphine after about 6 hours. In other embodiments, the sustained release formulation has an in vitro dissolution of about 75% to about 100% by weight nalbuphine from about 6 hours to about 8 hours. In further embodiments, the sustained release formulation has an in vitro dissolution of about 80% to about 100% by weight nalbuphine after about 12 hours. In still other embodiments, the sustained release formulation has an in vitro dissolution of about 80% to about 100% by weight nalbuphine from about 12 hours to about 24 hours. In some embodiments, the sustained release formulation has an in vitro dissolution of about 80% to about 100% after about 8 hours to about 12 hours. In yet other embodiments, the sustained release formulation has an in vitro dissolution of about 15% to about 75% by weight nalbuphine after about 1 hour. In still further embodiments, the sustained release formulation has an in vitro dissolution of about 50% by weight nalbuphine after about 1 hour. In some embodiments, the sustained release formulation has an in vitro dissolution of about 50% by weight nalbuphine after about 1 hour and about 75% to about 100% by weight nalbuphine from about 6 hours to about 8 hours. In some embodiments, the sustained release formulation has an in vitro dissolution of about 50% by weight nalbuphine after about 1 hour and about 75% to about 100% by weight nalbuphine from about 8 hours to about 12 hours. In some embodiments, the sustained release formulation has an in vitro dissolution of about 50% by weight nalbuphine after about 1 hour and about 75% to about 100% by weight nalbuphine from about 12 hours to about 24 hours. In some embodiments, the sustained release formulation has an in vitro dissolution of about 50% by weight nalbuphine after about 1 hour and about 80% to about 100% by weight nalbuphine after about 12 hours.

Where the tablet is a multilayer dosage form having a first extended release layer and a second, immediate release, layer, the sustained release formulation has an in vitro dissolution of about 25% to about 75% by weight nalbuphine after about 1 hour. In some embodiments, the multilayer dosage form has an in vitro dissolution of about 25% by weight nalbuphine after about 1 hour. In some embodiments, the multilayer dosage form has an in vitro dissolution of about 50% by weight nalbuphine after about 1 hour. In some embodiments, the multilayer dosage form has an in vitro dissolution of about 75% to about 100% nalbuphine after about 6-8 hours. In some embodiments, the multilayer dosage form has an in vitro dissolution of about 75% to about 100% nalbuphine after about 8-12 hours. In some embodiments, the multilayer dosage form has an in vitro dissolution of about 75% to about 100% nalbuphine after about 12-24 hours. In some embodiments, the multilayer dosage form has an in vitro dissolution of about 75% to about 100% nalbuphine after about 12 hours.

In some embodiments, when administered orally to patients having either normal or impaired (e.g., reduced) kidney function, the sustained release formulations described herein exhibit the following in vivo characteristics: (a) a peak plasma level of nalbuphine occurs within about 4 hours to about 6 hours, e.g., for patients with uremic pruritus or renal impairment, or about 3 hours to about 5 hours, e.g., for patients without renal impairment after administration; (b) onset of nalbuphine anti-pruritic effect from about 30 minutes of dosing to within about 6 hours of dosing; (c) duration of the nalbuphine anti-pruritic effect is about 2 to about 24 hours; and (d) the relative nalbuphine bioavailability is about 0.5, about 1, about 1.5 or between about 0.5 to about 1.5 compared to an orally administered aqueous solution of nalbuphine. The time of onset for an anti-pruritic effect can depend on at least on dosing and the severity of pruritic symptoms. In some embodiments, the duration of the nalbuphine anti-pruritic effect is at least about 8 hours. In some embodiments, the duration of the nalbuphine anti-pruritic effect is at least about 9 hours. In some embodiments, the duration of the nalbuphine anti-pruritic effect is at least about 10 hours. In some embodiments, the duration of the nalbuphine anti-pruritic effect is at least about 11 hours. In some embodiments, the duration of the nalbuphine anti-pruritic effect is at least about 12 hours. In some embodiments, the duration of nalbuphine anti-pruritic effect is about 6, hours, 8 hours, 10 hours, 12 hours, 15 hours, or 18 hours. In some embodiments, the relative nalbuphine bioavailability is about 0.94 compared to an orally administered aqueous solution of nalbuphine. In some embodiments, the relative nalbuphine bioavailability is about 1.35 compared to an orally administered aqueous solution of nalbuphine.

In some embodiments, the sustained release nalbuphine formulations provide an oral unit dosage form including nalbuphine or a pharmaceutically acceptable salt, solvate or ester thereof. The oral dosage form provides an anti-pruritic effect over a period of at least about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours. In some embodiments, the oral dosage form provides an anti-pruritic effect over a period of about 6-18 hours, about 8-16 hours, about 8-12 hours, about 8 to about 24 hours, about 12 to about 24 hours, about 18 to about 24 hours, or about 8-10 hours. The oral dosage form provides an anti-pruritic effect over a period of about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours or about 24 hours.

In one embodiment, the oral dosage form provides an anti-pruritic effect as well as breaking the cycle effect, e.g., the itchy sensation does not return after certain treatment period.

In some embodiments, the oral dosage form provides a blood plasma level of nalbuphine characterized by one or more peaks followed by a plateau region. The plateau region is characterized as having a relatively consistent blood plasma level of nalbuphine (e.g., the blood plasma level of nalbuphine does not consistently increase or decrease from time point to time point). In some embodiments, the plateau region is characterized as having a consistent average blood plasma level of nalbuphine. The plateau region is contrasted with the region following the plateau region, in which the blood plasma level of nalbuphine generally decreases from one time point to the next. In some embodiments, the plateau region has a duration of at least about 1 hour, about 2 hours, about 3 hours, about 4 hours, about 5 hours, about 6 hours, about 7 hours, about 8 hours, about 9 hours, about 10 hours, about 11 hours or about 12 hours. In some embodiments, the plateau region has a duration from about 1 hour to about 12 hours, from about 2 hours to about 10 hours, from about 2 hours to about 8 hours, from about 2 hours to about 7 hours or from about 4 hours to about 10 hours, from about 4 hours to about 8 hours, or from about 4 hours to about 6 hours. In some embodiments, the blood plasma level of nalbuphine at each time point in the plateau region ranges from about 75% to about 125% of the mean blood plasma level in the plateau region. In some embodiments, the blood plasma level of nalbuphine at each time point in the plateau region ranges from about 80% to about 120% of the mean blood plasma level in the plateau region. In some embodiments, the blood plasma level of nalbuphine at each time point in the plateau region ranges from about 85% to about 115% of the mean blood plasma level in the plateau region. In some embodiments, the blood plasma level of nalbuphine at each time point in the plateau region ranges from about 90% to about 110% of the mean blood plasma level in the plateau region.

In some embodiments, the minimum blood plasma level of nalbuphine observed during the plateau region is not more than about 25% below the mean blood plasma level for all time points in the plateau region. In some embodiments, the minimum blood plasma level of nalbuphine observed during the plateau region is not more than about 20% below the mean blood plasma level in the plateau region. In some embodiments, the minimum blood plasma level of nalbuphine observed during the plateau region is not more than about 15% below the mean blood plasma level in the plateau region. In some embodiments, the minimum blood plasma level of nalbuphine observed during the plateau region ranges from about 75% to about 100% of the mean blood plasma level in the plateau region. In some embodiments, the minimum blood plasma level of nalbuphine observed during the plateau region ranges from about 80% to about 100% of the mean blood plasma level in the plateau region. In some embodiments, the minimum blood plasma level of nalbuphine observed during the plateau region ranges from about 85% to about 100% of the mean blood plasma level in the plateau region. In some embodiments, the minimum blood plasma level of nalbuphine observed during the plateau region ranges from about 80% to about 95% of the mean blood plasma level in the plateau region.

### Co-Therapy

While the compositions can be administered as the sole active pharmaceutical ingredient or sole active anti-pruritus ingredient in the methods described herein, in other embodiments they can also be used in combination with one or more ingredients which are known to be therapeutically effective against pruritus and/or compliment the effect of anti-pruritus ingredient.

For example, in some embodiments, the present methods can employ nalbuphine or a pharmaceutically acceptable salt, solvate or ester thereof in conjunction with one or more anti-pruritic agents. In some embodiments, additional compounds combined with the anti-pruritic agent, e.g., nalbuphine, or a pharmaceutically acceptable salt, solvate or ester thereof, include antihistamines, anti-inflammatory corticosteroids, topical anti-infectives and antifungals, antibacterials, and antivirals, cytotoxic agents, and counter-irritants/analgesics. Other antipruritic agents include anti-depressants, vitamin D, kappa agonists, irritants such as coal tar derivatives and psoralens, 5-HT3 antagonists such as ondansetron, H2 receptor antagonist such as cimetidine, H1 receptor antagonist such as cetirizine, immunomodulators such as tacrolimus, immunosupressants such as cyclosporine A, μ-antagonists, capsaicin, cannabinoids, latex extracts from various Croton species found in the Amazon jungle (e.g., Zangrado®), or Nkl antagonists, etc. In some embodiments, nalbuphine or a pharmaceutically acceptable salt, solvate or ester thereof is not administered in combination with a second anti-pruritus agent, e.g., co-formulated or administered separately.

### Dosing

The invention provides methods for treating pruritus by administering an effective amount of an anti-pruritic agent, i.e., nalbuphine or a pharmaceutically acceptable salt, solvate or ester thereof, to a patient in need thereof. An effective amount is an amount sufficient to eliminate or significantly reduce pruritus symptoms or to alleviate those symptoms (e.g., reduce the symptoms, such as itching, compared to the symptoms present prior to treatment). Formulations employed in the present methods can incorporate the anti-pruritic agent in a sustained release formulation such that the formulation provides therapeutically effective blood plasma levels of nalbuphine for the treatment of pruritus.

According to some embodiments of the present invention, administering of nalbuphine or a pharmaceutically acceptable salt, solvate or ester thereof provides statistically significant therapeutic effect. In one embodiment, the statistically significant therapeutic effect is determined based on one or more standards or criteria provided by one or more regulatory agencies in the United States, e.g., FDA or other countries. In another embodiment, the statistically significant therapeutic effect is determined based on results obtained from regulatory agency approved clinical trial set up and/or procedure.

In some embodiments, the statistically significant therapeutic effect is determined based on a patient population of at least 50, 60, 100, 200, 300, 400, 500, 600, 700, 800, 900, 1000 or 2000. In some embodiments, the statistically significant therapeutic effect is determined based on data obtained from randomized and double blinded clinical trial set up. In some embodiments, the statistically significant therapeutic effect is determined based on data with a p value of less than or equal to about 0.05, 0.04, 0.03, 0.02 or 0.01. In some embodiments, the statistically significant therapeutic effect is determined based on data with a confidence interval greater than or equal to 95%, 96%, 97%, 98% or 99%. In some embodiments, the statistically significant therapeutic effect is determined on approval of Phase III clinical trial of the methods provided by the present invention, e.g., by FDA in the US.

In some embodiments, the statistically significant therapeutic effect is determined by a randomized double blind clinical trial of patients treated with nalbuphine or a pharmaceutically acceptable salt, solvate or ester thereof and optionally in combination with standard care. In some embodiment, the statistically significant therapeutic effect is determined by a randomized clinical trial and using Numerical Rating Scale (NRS) as primary efficacy parameter and optionally in combination with any other commonly accepted criteria for pruritus assessment.

In general, statistical analysis can include any suitable method permitted by a regulatory agency, e.g., FDA in the US or Europe or any other country. In some embodiments, statistical analysis includes non-stratified analysis, log-rank analysis, e.g., from Kaplan-Meier, Jacobson-Truax, Gulliken-Lord-Novick, Edwards-Nunnally, Hageman-Arrindel and Hierarchical Linear Modeling (HLM) and Cox regression analysis.

According to the present invention, the anti-pruritic agent is administered on a once or twice a day basis to provide effective relief of the symptoms of prurigo nodularis. In some embodiments, a total daily dose is about 60 mg, about 90 mg, about 120 mg, about 180 mg, about 240 mg, about 360 mg, or about 480 mg. In some embodiments, the total daily dose of the anti-pruritic agent can be at least about 180 mg a day for the treatment of prurigo nodularis. In some embodiments, the total daily dose of the anti-pruritic agent can be at least about 360 mg a day for the treatment of prurigo nodularis. In some embodiments, the total daily dose of the anti-pruritic agent can be about 180 mg a day for the treatment of prurigo nodularis. In some embodiments, the total daily dose of the anti-pruritic agent can be about 360 mg a day for the treatment of prurigo nodularis.

In some embodiments, about 90 mg of the anti-pruritus agent twice a day is selected to provide a substantial reduction in itch for patients with prurigo nodularis. In some embodiments, about 180 mg of the anti-pruritus agent once a day is selected to provide a substantial reduction in itch for patients with prurigo nodularis. In some embodiments, about 180 mg of the anti-pruritus agent twice a day is selected to provide a substantial reduction in itch for patients with prurigo nodularis. In some embodiments, about 360 mg of the anti-pruritus agent once a day is selected to provide a substantial reduction in itch for patients with prurigo nodularis. In particular embodiments, about 180 mg of the anti-pruritus agent twice a day is selected to provide a reduction of chronic itch in patients with prurigo nodularis (PN).

Reduction of itch in patients with pruritic conditions can be determined by various methods. In some embodiments, the effectiveness of a dosage regimen can be determined by evaluation via a Pruritus Visual Analog Scale (VAS) test, such as the worst-itch VAS. In some embodiments, the effectiveness of a dosage regimen can be determined by evaluation via a worst or average itching intensity Numerical Rating Scale (NRS). In yet some other embodiments, the effectiveness of a dosage regimen can be determined by evaluation via a worst or average itching intensity Numerical Rating Scale (NRS), an MOS Sleep Scale-Revised (MOS Sleep-R) scale, a Hospital Anxiety and Depression Scale (HADS), a Patient Global index scale, a Global Physician index scale, Patient Benefit Index—pruritus version (PBI-P), Prurigo Activity Score (PAS), itchy, burning and stinging Verbal Rating Scale (VRS) score, Itchy Quality of Life (ItchyQoL) (Emory University; http://emoryott.technologypublisher.com/tech?title=ItchyQol %3a_A_Pruritus-Specific_Quality_of Life_Instrument), Patient Global Assessment (PGA) via ItchApp, vPGA, Dermatology Life Quality Index (DLQI), Nocturnal scratching using actigraphy, nerve fiber density and MOR/KOR density, Beck Depression Index, or any combination thereof. In still another embodiment, the effectiveness of a dosage regimen can be determined by evaluation via a worst or average itching intensity NRS as a primary efficacy endpoint in association with secondary efficacy endpoints such as an MOS Sleep Scale-Revised (MOS Sleep-R) scale, a Hospital Anxiety and Depression Scale (HADS), a Patient Global index scale, a Global Physician index scale, Patient Benefit Index—pruritus version (PBI-P), Prurigo Activity Score (PAS), itchy, burning and stinging Verbal Rating Scale (VRS) score, Itchy Quality of Life (ItchyQoL), Patient Global Assessment (PGA) via ItchApp, vPGA, Dermatology Life Quality Index (DLQI), Nocturnal scratching using actigraphy, nerve fiber density and MOR/KOR density, Beck Depression Index, or any combination thereof.

According to some embodiments of the present invention, the dosing frequency and dose amount per administration of the anti-pruritus agent are selected to provide therapeutic effects for the treatment of pruritus. In certain embodiments, nalbuphine or a pharmaceutically acceptable salt, solvate or ester thereof is administered on a once-a-day or twice-a-day basis for at least a week, for example, about a week, about 2 weeks, about 3 weeks, about 4 weeks, about 5 weeks, about 6 weeks, about 7 weeks, about 8 weeks, about 9 weeks, about 10 weeks, about 12 weeks, about 18 weeks, about 24 weeks, and about 50 weeks. In certain embodiments, at least about 90 mg or about 90 mg of nalbuphine or a pharmaceutically acceptable salt, solvate or ester thereof is administered on a once-a-day or twice-a-day basis for at least a week. In certain embodiments, at least about 180 mg or about 180 mg of nalbuphine or a pharmaceutically acceptable salt, solvate or ester thereof is administered on a once-a-day or twice-a-day basis for at least a week. In certain embodiments, at least about 360 mg or about 360 mg of nalbuphine or a pharmaceutically acceptable salt, solvate or ester thereof is administered on a once-a-day or twice-a-day basis for at least a week.

In some embodiments, after the treatment the patient experiences a substantial reduction of itch that is characterized by at least about a 30% decline in worst or average itching intensity Numerical Rating Scale (NRS) value compared to prior to the treatment. In some embodiments, the reduction of itch is characterized by a decline in NRS value ranging from about 30% to about 100%, for example, about 30%, about 40%, about 50%, about 60%, about 70%, about 80%, about 90%, and about 100%, compared to prior to the treatment.

In some embodiments, after the treatment the patient experiences a substantial reduction of itch that is characterized by at least about a 10% improvement in Itchy Quality of Life (ItchyQoL) scale compared to prior to the treatment. In some embodiments, the reduction of itch is characterized by an improvement in Itchy Quality of Life (ItchyQoL) scale ranging from about 10% to about 100%, for example, about 10%, about 20%, about 30%, about 40%, about 50%, about 60%, about 70%, about 80%, about 90%, and about 100%, compared to prior to the treatment.

In some embodiments, after the treatment the patient experiences a substantial reduction of itch that is characterized by at least about a 20% improvement in MOS Sleep Scale-Revised (MOS Sleep-R) scale or any of the respective subscale analysis of sleep disturbance, snoring, shortness of breath or headache, sleep somnolence, sleep quality or the Sleep Problems Index I or Sleep Problems Index II, compared to prior to the treatment. In some embodiments, the reduction of itch is characterized by an improvement in MOS Sleep Scale-Revised (MOS Sleep-R) scale or any of the respective subscale ranging from about 10% to about 100%, for example, about 10%, about 20%, about 30%, about 40%, about 50%, about 60%, about 70%, about 80%, about 90%, and about 100%, compared to prior to the treatment.

In some embodiments, after the treatment the patient experiences a substantial reduction of itch that is characterized by at least about a 10% improvement in Hospital Anxiety and Depression Scale (HADS) sleep scale compared to prior to the treatment. In some embodiments, the reduction of itch is characterized by an improvement in Hospital Anxiety and Depression Scale (HADS) ranging from about 10% to about 100%, for example, about 10%, about 20%, about 30%, about 40%, about 50%, about 60%, about 70%, about 80%, about 90%, and about 100%, compared to prior to the treatment.

In some embodiments, after the treatment the patient experiences a substantial reduction of itch, burning sensation, and/or stinging sensation that is characterized by at least about a 10% improvement in itchy, burning and/or stinging Verbal Rating Scale (VRS) score compared to prior to the treatment. In some embodiments, the reduction of itch is characterized by an improvement in itchy, burning and/or stinging Verbal Rating Scale (VRS) score ranging from about 10% to about 100%, for example, about 10%, about 20%, about 30%, about 40%, about 50%, about 60%, about 70%, about 80%, about 90%, and about 100%, compared to prior to the treatment.

In some embodiments, after the treatment the patient experiences a substantial reduction of itch that is characterized by at least about a 10% improvement in Patient Benefit Index—pruritus version (PBI-P) scale compared to prior to the treatment. In some embodiments, the reduction of itch is characterized by an improvement in Patient Benefit Index—pruritus version (PBI-P) scale ranging from about 10% to about 100%, for example, about 10%, about 20%, about 30%, about 40%, about 50%, about 60%, about 70%, about 80%, about 90%, and about 100%, compared to prior to the treatment.

In some embodiments, after the treatment the patient experiences a substantial reduction of itch that is characterized by at least one category/stage improvement in Prurigo Activity Score (PAS). In particular embodiments, after the treatment the patient experiences a reduction of itch that results in at least one category/stage improvement in Prurigo Activity Score (PAS) domains of number of prurigo lesions, prurigo lesions with excoriations/crusts and/or healed prurigo lesions. The PAS consists of 7 qualitative and quantitative measurements related to the examination of the skin. Type, number, distribution, affected body parts, and quantitative number of lesions in a representative body part are documented. The biggest lesion and the most representative lesion are monitored with documentation of height and area measurements. Prurigo lesion activity is recorded as a percentages based on their stage (0-4). In some embodiments, number measurements of lesions on the body have four numerical categories: 0, 1-19, 20-100 and >100. In some embodiments, prurigo lesions with excoriations/crusts have five categories: Stage 4 (76%-100%), Stage 3 (51%-75%), Stage 2 (26%-50%), Stage 1 (1-25%) and Stage 0 (0%). In some embodiments, healed prurigo lesions have five categories: Stage 4 (0-24%), Stage 3 (25-49%), Stage 2 (50-74%), Stage 1 (75%-99%), Stage 0 (100%).

In some embodiments, the daily dose of the anti-pruritic agent is in a once or twice daily dose, and then titrated upward until the patient experiences satisfactory relief from the pruritic condition. The daily dose can be titrated in increments ranging from about 5 mg to about 360 mg (e.g., about 15 mg, about 30 mg or about 60 mg). The daily dose can be titrated in one or more steps. The daily dosage can be titrated by increasing a single daily dosage, or each dose of a twice-daily dosing regimen. The amount a dosage is stepped, where there are multiple titration steps, can be the same, or can be different.

In some embodiments, the titration may be initiated with about 15 mg, about 30 mg or about 60 mg of the anti-pruritic agent once or twice daily. In certain embodiments, doses can be adjusted in 30 mg increments every 1 to 4 days. Patients can self-titrate to effect over from about 7 days to about 30 days (for example, from about 12 days to about 20 days) to a dose that provides adequate relief from itch and minimizes adverse reactions. In some embodiments, the titration is conducted for at least about one week, 2 weeks, 3 weeks, 4 weeks or 5 weeks until a steady state is achieved in the patient.

In certain embodiments, patients can be provided initially with 15 mg, 30 mg or 60 mg tablets to self-titrate to effect up to about 60 mg, about 90 mg, about 120 mg, about 180 mg, about 240 mg, about 360 mg, or about 480 mg once or twice a day. In some embodiments, the titration dose is started with about 15 mg or about 30 mg, and then gradually increased to about 90 mg or 180 mg twice a day, e.g., for patients with prurigo nodularis. In another embodiment, the titration dose is started with about 15 mg or about 30 mg, and then gradually increased to about 180 mg or 360 mg once a day, e.g., for patients with prurigo nodularis.

In particular embodiments, the anti-pruritic agent is nalbuphine and the titration is conducted for two weeks according to the dose schedule provided in the following table:

| Day | AM dosage (mg) | PM dosage (mg) |
|---|---|---|
| Day 1 | 0 | 30 |
| Day 2 | 0 | 30 |
| Day 3 | 30 | 30 |
| Day 4 | 30 | 30 |
| Day 5 | 30 | 60 |
| Day 6 | 60 | 60 |
| Day 7 | 60 | 60 |
| Day 8 | 60 | 90 |
| Day 9 | 90 | 90 |
| Day 10 | 90 | 90 |
| Day 11 | 90 | 120 |
| Day 12 | 120 | 120 |
| Day 13 | 120 | 120 |
| Day 14 | 120 | 180 |

According to some embodiments of the present invention, the methods of the present invention provide therapeutically effective blood plasma levels of nalbuphine for treating prurigo nodularis. Blood plasma levels of nalbuphine may be expressed using pharmacokinetic parameters that are known to those skilled in the art, such as steady state plasma levels, AUC, Cmax and Cmin.

In some embodiments, the present methods provide steady state plasma levels of nalbuphine that correlate to one or more statistically significant therapeutic effects. In certain embodiments, the therapeutically effective steady state plasma levels of nalbuphine provided by the methods of the present invention range from about 10 ng/mL to about 80 ng/mL, including about 20 ng/mL, about 25 ng/mL, about 30 ng/mL, about 35 ng/mL, about 40 ng/mL, about 45 ng/mL, about 50 ng/mL, about 55 ng/mL, about 60 ng/mL, about 65 ng/mL, about 70 ng/mL, about 75 ng/mL and about 80 ng/mL, including all ranges there between. In certain embodiments, the therapeutically effective steady state plasma levels of nalbuphine is provided by administering a daily dose of nalbuphine or a pharmaceutically acceptable salt or ester is about 360 mg. In further embodiments, the therapeutically effective steady state plasma levels of nalbuphine is provided by administering about 180 mg of nalbuphine or a pharmaceutically acceptable salt or ester thereof twice a day.

In some embodiments, the present methods provide mean steady state AUC_{0-24 h} (expressed in terms of ng*hr/mL) levels of nalbuphine that correlate to one or more statistically significant therapeutic effects. In certain embodiments, the therapeutically effective mean steady state AUC 0-24 h levels of nalbuphine provided by the methods of the present invention range from about 200 ng*hr/mL to about 1600 ng*hr/mL, including about 300 ng*hr/mL, about 400 ng*hr/mL, about 500 ng*hr/mL, about 600 ng*hr/mL, about 700 ng*hr/mL, about 800 ng*hr/mL, about 900 ng*hr/mL, about 1000 ng*hr/mL, about 1100 ng*hr/mL, about 1200 ng*hr/mL, about 1300 ng*hr/mL, about 1400 ng*hr/mL, and about 1500 ng*hr/mL, including all ranges there between. In certain embodiments, the therapeutically effective mean steady state AUC 0-24 h levels of nalbuphine is provided by administering a daily dose of nalbuphine or a pharmaceutically acceptable salt or ester is about 360 mg. In further embodiments, the therapeutically effective mean steady state AUC 0-24 h levels of nalbuphine is provided by administering about 180 mg of nalbuphine or a pharmaceutically acceptable salt or ester thereof twice a day.

In certain embodiments, the anti-pruritus agent is nalbuphine, and the metabolites include glucuronides (most likely on the phenol and cyclohexane rings), two hydroxylated nalbuphine metabolites (on the cyclobutane ring) and three ketones (hydroxylation of the cyclobutane ring, followed by oxidation to a carbonyl or followed by ring opening of the cyclobutane ring). In some embodiments, the nalbuphine metabolites include nalbuphine 3-glucuronide or 6-glucuronide. In some other embodiments, the nalbuphine metabolites include triple hydroxylated nalbuphine, mono-hydroxylated nalbuphine, or mono-glucuronidated nalbuphine or a combination thereof. In certain embodiments, the one or more metabolites of the anti-pruritus agent do not have detectable anti-pruritus activity. In other embodiments, one or more of the metabolites of the anti-pruritus agent exhibit anti-pruritus activity.

In embodiments wherein one or more metabolites of the anti-pruritus agent exhibit anti-pruritus activity, the dosing regimen of the anti-pruritus agent may be adjusted and/or titrated as described hereinabove depending on the clearance rate of the one or more metabolites exhibiting anti-pruritic activity. Such dosage adjustment and/or titration of the dosage of the anti-pruritic agent can be performed to prevent accumulation of either the anti-pruritic agent and/or one or more metabolites, which can also exhibit anti-pruritic activity, to avoid toxicity effects in a patient treated with the present anti-pruritic agent.

In some embodiments, the anti-pruritus agent is completely metabolized (e.g., about 100% metabolized). In other embodiments, the anti-pruritus agent is not completely metabolized (e.g., less than about 100% metabolized). For example, in some embodiments, the anti-pruritus agent is about 100% metabolized, about 95% metabolized, about 90% metabolized, about 85% metabolized, about 80% metabolized, about 75% metabolized, about 70% metabolized, about 65% metabolized, about 60% metabolized, about 55% metabolized, about 50% metabolized, about 45% metabolized, about 40% metabolized, about 35% metabolized, about 25% metabolized, about 20% metabolized, about 15% metabolized, about 10% metabolized, about 5% metabolized, about 1% metabolized, or about 0% metabolized. In certain embodiments, the amount of dialyzable agent can be measured or monitored by the level of accumulation, e.g., blood plasma level of the anti-pruritus agent or one or more of its metabolites.

The embodiments described herein should be understood to be illustrative of the present invention, and should not be construed as limiting. On the contrary, the present disclosure embraces alternatives and equivalents thereof, as embodied by the appended claims. Each reference disclosed herein is incorporated by reference herein in its entirety.

The following non-limiting examples illustrate various aspects of the present invention.

### EXAMPLES

### Example 1

A 30 mg, 60 mg or 180 mg extended release (ER) nalbuphine tablet was prepared as follows: Nalbuphine HCl, mannitol, xanthan gum, locust bean gum and calcium sulfate dihydrate were added to a high shear mixer and dried mix at low speed. A granulating solution (water for injection or purified water) was introduced into the mixer at low speed. The wet granulation was granulated at high speed and dried in a fluid bed processor. The dried granules were milled and sized using a conventional mill. The milled granulation was transferred into a diffusion (tumble) mixer. Hydroxypropylcellulose and, when applicable, fumaric acid (180 mg formulations only) were added to the diffusion mixer and blended. Thereafter, magnesium stearate was added to the diffusion mixer and blended. The final blend was compressed using a rotary tablet press. Tablets may be coated with a non-functional Opadry white coating.

TABLE 1
30 mg Extended Release Nalbuphine Tablet

| Ingredient | mg/tablet |
|---|---|
| Nalbuphine HCI | 30.0 |
| Mannitol | 108.0 |
| Hydroxypropylcellulose | 35.0 |
| Locust bean gum | 32.4 |
| Xanthan gum | 21.6 |
| Calcium sulfate dehydrate | 18.0 |
| Magnesium stearate | 1.9 |
| Water for injection or Purified water | QS |
| Total: | 246.9 |

The tablets were coated with a non-functional coat (Table 2).

TABLE 2
Nalbuphine HCl ER Tablets, 30 mg, 60 mg, or 180 mg Composition

| Component | Tablet (mg/tablet) |
|---|---|
| Nalbuphine HCl | 30.0 |
| Mannitol | 108.0 |
| Hydroxypropylcellulose | 35.0 |
| Locust bean gum | 32.4 |
| Xanthan gum | 21.6 |
| Calcium sulfate dihydrate | 18.0 |
| Magnesium stearate | 1.9 |
| Opadry II White | 7.4 |
| Sterile water for irrigation | QS |
| Total | 254.3 |
| Nalbuphine HCl | 60.0 |
| Mannitol | 72.0 |
| Hydroxypropylcellulose | 30.0 |
| Locust bean gum | 21.6 |
| Xanthan gum | 14.4 |
| Calcium sulfate dihydrate | 12.0 |
| Magnesium stearate | 1.6 |
| Opadry II White | 6.355 |
| Sterile water for irrigation | QS |
| Total | 218 |
| Nalbuphine HCl | 180 |
| Mannitol | 160.8 |
| Hydroxypropylcellulose | 59.6 |
| Locust bean gum | 48.2 |
| Fumaric acid | 24.8 |
| Xanthan gum | 32.2 |
| Calcium sulfate dihydrate | 26.8 |
| Magnesium stearate | 4.0 |
| Sterile water for irrigation | QS |
| Total | 534.9 |

### Example 2

This clinical study was a double-blind, placebo-controlled trial in which prurigo nodularis patients were randomized in a 1:1:1 ratio to nalbuphine ER tablets to a target dose of 90 mg twice daily (BID), nalbuphine HCl ER tablets 180 mg BID, or placebo tablets BID. Use of nonsedating rescue antihistamine medications (non-first generation H1-antihistamines [Simons F E. Advances in H1-antihistamines. N Engl J Med. 2004; 351(21):2203-17.]) were allowed in the study but their use was recorded. Cleansing emollients were allowed for hygiene purposes provided there were no active substances (such as menthol or urea) contained in the lotion. A placebo comparator was chosen because there are no approved treatments for prurigo nodularis in the United States or Europe or an established standard of care that could serve as an active control.

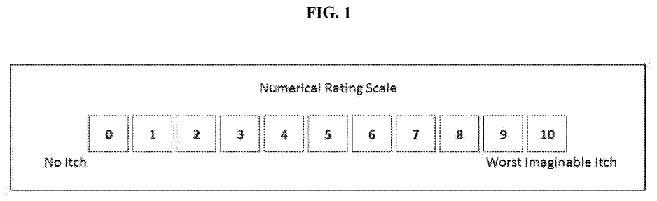

The primary objectives were to evaluate the effects of two doses of nalbuphine HCl ER tablets on the 7-day average daily worst itch (i.e., most severe) intensity using a Worst Itching Intensity Numerical Rating Scale (NRS, 0 [no itching]-10 [worst possible itching]; range, 0-10; anchors at 0 “no itching”; 4-6 “moderate itching”; and 10 “worst possible itching”) (FIG. 1) as well as safety and tolerability. The study was conducted at four North American sites and four European sites. The Sponsor oversaw the conduct of the trial and an independent unblinded Data Safety Monitoring Board reviewed safety data approximately once a month during the conduct of the trial.

### Participants

To be eligible, patients had to suffer from prurigo nodularis; patients had to have generalized PN; and patients had to have the NRS based worst itch recorded daily over the 7 contiguous days and must have at least five measurements recorded. The mean value of the measurements must be ≥5.

### Inclusion Criteria

Patients were required to meet all of the following criteria to be eligible for inclusion in the study:
1) Individuals suffering from PN (definition: presence of pruritic nodules and/or papules due to chronic pruritus, i.e., pruritus and PN was actively present for at least 6 weeks prior to randomization).
2) Aged 18 years and older at the time of consent.
3) Generalized PN defined as PN lesions involving 2 distinct anatomical areas: for example, either 2 limbs; or a single limb and some axial portion of the body. The patient was also eligible with only axial lesions with 2 distinct anatomical areas of involvement that had no peripheral nervous system overlap: for example, lesions involving a portion of the cranium and a portion of the trunk of the body. For purposes of this study, the axial portion was defined as any nonappendicular portion of the body.
4) Numerical rating scale-based worst itch (i.e., most severe) recorded daily over the 7 contiguous days prior to Visit 2 via daily e-diary had at least 5 measurements recorded. The mean value of the measurements was to be ≥5. Note: The last NRS score used in the calculation could be captured at Visit 2.
5) Agree to comply with the contraception requirements as below:

Female patients of childbearing potential were required to use 1 barrier method (e.g., condom, cervical cap, or diaphragm) of contraception in addition to 1 additional method (e.g., intrauterine device in place for at least 1 month, stable hormonal contraception for at least 3 months, or tubal ligation, Essure procedure, or spermicide). For female patients using a barrier method plus spermicide, that method must have been used for at least 14 days prior to screening.

Female patients who were abstinent could participate in the study, however; they were counseled on the requirement to use appropriate contraception if they became sexually active. This counseling occurred at each study visit and documented in source records.
6) Have been adequately informed of the nature and risks of the study and have given written informed consent prior to Screening.

### Exclusion Criteria

If a patient meets any of the following criteria, he or she is not eligible:
1) Chronic pruritus due the following conditions: localized PN (only 1 localization affected, e.g., only 1 arm), lichen amyloidosis, or peripheral segmental neuropathic pruritus (such as notalgia paresthetica, brachioradial pruritus)
2) Active dermatoses in need of treatment (such as atopic dermatitis, bullous pemphigoid) that had not evolved into the diagnosis of prurigo nodularis or other dermatologic conditions that in the opinion of the investigator could confound the ability to assess the NRS.
3) Major psychiatric disorder in the opinion of the investigator that could interfere with the assessment of study drug, such as delusional parasitosis.
4) Patients receiving treatment for human immunodeficiency virus infection.
5) Serum bilirubin >2.5 times upper limit of normal range at screening unless explained by a clinical diagnosis of Gilbert's syndrome.
6) Serum hepatic alanine aminotransferase or aspartate aminotransferase enzymes >2 times upper limit of normal range at screening.
7) Estimated glomerular filtration rate ≤44 mL/min/1.73 m2 at screening.
8) Use of the following medications:
Topical antihistamines (2 weeks prior to e-diary NRS and VRS collection during the screening period)
   Topical capsaicin (2 weeks prior to e-diary NRS and VRS collection during the screening period)
   Topical calcineurin inhibitors (2 weeks prior to e-diary NRS and VRS collection during the screening period)
   Topical antibiotics (2 weeks prior to e-diary NRS and VRS collection during the screening period)
   Topical steroids (2 weeks prior to e-diary NRS and VRS collection during the screening period)
   Antiseptic bathes and antiseptic cleansing lotions (2 weeks prior to e-diary NRS and VRS collection during the screening period)
   Systemic antihistamines (2 weeks prior to e-diary NRS and VRS collection during the screening period)
   Anti-convulsant class drugs (4 weeks prior to e-diary NRS and VRS collection during the screening period)
   Systemic steroids (4 weeks prior to e-diary NRS and VRS collection during the screening period)
   Naltrexone or naloxone (4 weeks prior to e-diary NRS and VRS collection during the screening period)
   Cyclosporine A and other immunosuppressants (4 weeks prior to e-diary NRS and VRS collection during the screening period)
   Antidepressant medications (4 weeks prior to e-diary NRS and VRS collection during the screening period)
   Benzodiazepine class drugs (2 weeks prior to e-diary NRS and VRS collection during the screening period)
   Neuroleptic class drugs (2 weeks prior to e-diary NRS and VRS collection during the screening period)

NOTE: During washout of excluded medications, rescue was permitted with cleansing lotion or pure emollients (emollients without active substances such as menthol, urea, etc.).
9) Ultraviolet therapy (Psoralen plus ultraviolet light A [PUVA], ultra violet A, ultra violet B, Excimer) (4 weeks prior to e-diary NRS and VRS collection during the screening period).
10) Patients who received opiates within 14 days prior to e-diary NRS and VRS collection during the screening period.
11) Patients with a history of congestive heart failure of Class 2 or higher as graded using the New York Heart Association scale.
12) Patients with a history of angina pectoris grade 2 or higher as graded using the Canadian Cardiovascular Society grading scale.
13) History of ventricular tachycardia, torsade de pointes, family history of sudden death, myocardial infarction or acute coronary syndrome within the previous 3 months, as reported by the patient.
14) Serum potassium below the laboratory lower limit of normality. Potassium supplementation could be prescribed and the serum potassium level repeated.
15) QTcF interval >450 ms on screening ECG.
16) Heart rate <50 beats per minute (bpm) on any screening measurement. Patients with a resting heart rate of <50 bpm had it repeated once after 5 minutes in the supine position, and if it remained <50 bpm during the repeat, they were considered a screen failure.
17) Use of a medication known to be associated with risk of torsade de pointes within 14 days prior to e-diary NRS and VRS collection during the screening period.
18) Significant medical condition or other factors that in the opinion of the investigator might have interfered with the conduct of the study.
19) Exposure to any investigational medication, including placebo, within 4 weeks of e diary NRS and VRS collection during the screening period.
20) Patients who had a confirmed malignant tumor and were receiving active treatment with a systemic drug (hormonal treatment could be acceptable to study enrollment if approved by the medical monitor).
21) History of substance abuse, as determined by the investigator.
22) Known hypersensitivity or allergy to nalbuphine or vehicle components.
23) Known drug allergy to opioids.
24) A pregnant or lactating female.

### Outcomes

The initial primary endpoint was the proportion of patients who achieved at least 30% reduction threshold in 7-day average daily diary-reported worst itch intensity NRS score from baseline to Week 10/last observed post-baseline value (LOV). For the primary endpoint, a responder was a patient who achieved at least 30% reduction threshold in 7-day average daily diary-reported worst itch intensity NRS score from baseline to Week 10/last observed post-baseline value (LOV).

Before study completion and unblinding, however, a more substantial 50% NRS reduction criterion was added as an important definition of response. For this criterion, a responder was a patient who achieved at least 50% reduction threshold in 7-day average daily diary-reported worst itch intensity NRS score from baseline to Week 10/last observed post-baseline value (LOV).

Secondary endpoints included:
(1) The proportion of patients with at least a 30% reduction in the 7-day average daily diary-reported NRS itch intensity from baseline to the evaluation visit (Week 10).
(2) The proportion of patients with at least a 50% reduction in the 7-day average daily diary-reported NRS itch intensity from baseline to the evaluation visit (Week 10).
(3) The mean week-by-week changes in 7-day average daily worst itch intensity and average daily itch intensity through the evaluation visit (Week 10) compared to baseline.
(4) The mean week-by-week changes in the individual 7-day average daily itchy, burning, and stinging Verbal Rating Scale (VRS) through the evaluation visit (Week 10) compared to baseline.
(5) Prurigo nodularis skin lesions, as quantitatively measured by the Prurigo Activity Score (PAS) recorded at baseline and during evaluation visit (Week 10).

Quality of life-related secondary endpoints included change from Day 1 to the Evaluation Period in itching-related quality of life (using the Itchy QoL) and itching-related sleep disruption (using the MOS Sleep Scale-R). The Hospital Anxiety and Depression Score (HADS) used as a general measure of anxiety and depression.

### Statistical Methods

The modified intent-to-treat (MITT) population consisted of all randomly assigned patients who provided a baseline calculated NRS and at least one post-baseline NRS during randomized treatment. Patients were analyzed according to the treatment to which they were randomly assigned.

The primary worst itch NRS endpoint was evaluated by the calculation of proportions, and statistical testing of each nalbuphine dose and placebo was undertaken using a stratified categorical method, the Cochran-Mantel-Haenszel test that took into account site. Specifically, each nalbuphine group was compared to placebo in the form of a stratified (by site) 2×2 table with a P value reported for the nalbuphine HCl ER 180 mg BID versus placebo comparison and a P value separately reported for the nalbuphine HCl ER 90 mg BID versus placebo comparison. The lower nalbuphine dose versus placebo P value was evaluated only if the higher nalbuphine dose versus placebo P value was statistically significant at the 0.05 level. Data from all post-baseline visits were used to fit the model.

Efficacy analysis of secondary continuous endpoints was performed using a mixed model repeated ANCOVA with the main effects of treatment and site, including the baseline value for each endpoint as the covariate. The model included time (i.e. Visit) as a factor variable and treatment*time (with placebo as the reference category) with an unstructured covariance structure for repeated measures. For other secondary efficacy endpoints, the Time factor corresponded to the scheduled study visits when the assessment was obtained (Visit 3, Visit 4, and Visit 5). Data from all post-baseline visits were used to fit the model.

Unless otherwise specified, “Baseline” for efficacy and safety information such as vital signs was defined as the last non-missing evaluation taken prior to the first dose of study drug (including repeated and unscheduled assessments). Baseline for e-diary-reported outcomes (e.g., worst intensity NRS) was derived as the average of the responses on the 7 days prior to the date of first dose of study drug.

### Interventions

In the first 2 weeks of treatment, patients were blindly force-titrated to their assigned target dose, with the patients in the active arms reaching a dose of 90 mg BID (NAL 90) and, for those in the high dose group, to 180 mg BID (NAL 180) in the second week. Study drug was administered in blister cards containing the labeled morning and evening doses for each day of the week. Subsequently, patients continued stable doses of the study drug for an additional 8 weeks and were then washed off study drug and followed for another 2 weeks for itching intensity and safety. Table 3 shows the dose schedule for the 90 mg BID, 180 mg BID and placebo groups.

TABLE 3

| | Day | Nalbuphine | Nalbuphine | |
|---|---|---|---|---|
| Study | of | 90 mg BID Dose | 180 mg BID Dose | |
| Week | Week | Arm | Arm | Placebo Arm |
| 1 | 1 | 30 mg (PM dose) | 30 mg (PM dose) | Placebo (PM |
| | | | | dose) |
| | 2 | Placebo AM; | Placebo AM; | Placebo BID |
| | | 30 mg PM | 30 mg PM | |
| | 3-4 | 30 mg BID | 30 mg BID | Placebo BID |
| | 5 | 30 mg AM; | 30 mg AM; | Placebo BID |
| | | 60 mg PM | 60 mg PM | |
| | 6-7 | 60 mg BID | 60 mg BID | Placebo BID |
| 2 | 1 | 60 mg AM; | 60 mg AM; | Placebo BID |
| | | 90 mg PM | 90 mg PM | |
| | 2-3 | 90 mg BID | 90 mg BID | Placebo BID |
| | 4 | 90 mg BID | 90 mg AM; | Placebo BID |
| | | | 120 mg PM | |
| | 5-6 | 90 mg BID | 120 mg BID | Placebo BID |
| | 7 | 90 mg BID | 120 mg AM; | Placebo BID |
| | | | 180 mg PM | |
| 3-10 | 1-7 | 90 mg BID | 180 mg BID | Placebo BID |

No down-titration was permitted during the study, although patients who missed 6 or more consecutive doses during the Stable Dose Period (Weeks 3-10) could, with the Medical Monitor's approval, re-start treatment with blinded re-titration of just the morning or evening dose for 3 days before returning to the original dose. Patients were allowed to remain on their background antipruritic medications such as antihistamines and the use of these medications and indication for treatment was collected.

### Sample Size Calculations

The sample size of 20 per treatment arm (in the modified intent-to-treat population) was based on 90% power and two-sided significance testing at the a α=0.05 level was sufficient to detect an active treatment response proportion of, for example, at least 80% versus a placebo response proportion of no more than 30%, where response was defined as at least a 30% reduction in the baseline NRS measure.

### Randomization

Randomization was performed by an interactive web-based computer system (IWRS). Upon confirmation of eligibility by the study site, patients were randomly assigned in a 1:1:1 ratio to 1 of 3 treatment groups: (1) Nalbuphine HCl ER tablets: 90 mg BID target dose; (2) Nalbuphine HCl ER tablets: 180 mg BID target dose and (3) Placebo tablets BID.

### Results

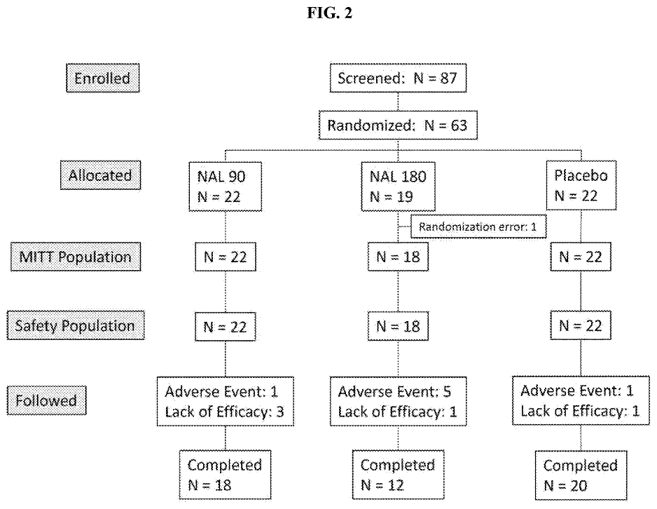

A total of 87 patients were screened, 63 patients were randomized (22 patients in the nalbuphine 90 mg treatment group, 19 patients in the nalbuphine 180 mg treatment group, and 22 patients in the placebo treatment group) and a total of 62 patients were treated in the study (FIG. 2). There were no notable differences in demographics and baseline characteristics in patients across treatment groups (Table 4).

TABLE 4

| | Nalbuphine | | Nalbuphine | | | |
|---|---|---|---|---|---|---|
| | 90 mg | | 180 mg | | Placebo | |
| | (N = 22) | | (N = 18) | | (N = 22) | |
| | n (%) | | n (%) | | n (%) | |
| Age (years) | | | | | | |
| Mean (SD) | 55.0 | (8.6) | 49.1 | (16.3) | 53.3 | (16.7) |
| Median | 57.0 | | 49.5 | | 55.5 | |
| Min, Max | 32, 69 | | 23, 72 | | 20, 75 | |
| Sex n (%) | | | | | | |
| Male | 10 | (45.5) | 8 | (44.4) | 10 | (45.5) |
| Female | 12 | (54.5) | 10 | (55.6) | 12 | (54.5) |
| Race n (%) | | | | | | |
| White | 20 | (90.9) | 15 | (83.3) | 19 | (86.4) |
| Black or | 2 | (9.1) | 2 | (11.1) | 2 | (9.1) |
| African | | | | | | |
| American | | | | | | |
| Asian | 0 | | 1 | (5.6) | 0 | |
| American | 0 | | 0 | | 0 | |
| Indian or | | | | | | |
| Alaska | | | | | | |
| Native | | | | | | |
| Native | 0 | | 0 | | 0 | |
| Hawaiian | | | | | | |
| or other Pacific | | | | | | |
| Islander | | | | | | |
| Other | 0 | | 0 | | 1 | (4.5) |
| Ethnicity | | | | | | |
| n (%) | | | | | | |
| Hispanic or | 0 | | 0 | | 0 | |
| Latino | | | | | | |
| Not Hispanic | 22 | (100) | 18 | (100) | 22 | (100) |
| or Latino | | | | | | |
| Missing | 0 | | 0 | | 0 | |
| Geographic | | | | | | |
| region n (%) | | | | | | |
| United States | 5 | (22.7) | 4 | (22.2) | 6 | (27.3) |
| Europe | 17 | (77.3) | 14 | (77.8) | 16 | (72.7) |
| Height (cm) | | | | | | |
| Mean (SD) | 171.34 | (8.97) | 172.68 | (6.85) | 170.91 | (10.29) |
| Median | 168.50 | | 170.00 | | 169.00 | |
| Min, Max | 157.5, 193.0 | | 164.0, 186.0 | | 154.9, 189.0 | |
| Weight (kg) | | | | | | |
| Mean (SD) | 85.06 | (20.29) | 79.10 | (21.47) | 87.03 | (20.92) |

| | | | |
|---|---|---|---|
| Median | 83.30 | 73.75 | 85.20 |
| Min, Max | 58.5, 129.0 | 54.5, 136.1 | 56.0, 136.5 |

The primary efficacy endpoint was the proportion of patients who achieved at least 30% reduction threshold in 7-day average daily diary-reported worst itch intensity NRS score from baseline to Week 10/LOV (i.e., proportion of responders) for each dose of nalbuphine, separately and placebo in the MITT population and is presented in Table 5. For LOV patients, no statistical differences were observed. For patients who completed the 10 week study, statistically significant differences in the proportion of responders were noted between the nalbuphine 180 mg treatment group and placebo treatment group (P=0.026).

TABLE 5

| | | |
|---|---|---|
| Nalbuphine | Nalbuphine | |
| 90 mg | 180 mg | Placebo |
| N (%) | N (%) | N (%) |

Patients meeting responder criterion at

| | | | |
|---|---|---|---|
| Week 10—LOV | 22 | 18 | 22 |
| Responders | 6 (27.3) | 8 (44.4) | 8 (36.4) |
| P value | 0.779 | 0.323 | — |

Patients meeting responder criterion at

| | | | |
|---|---|---|---|
| Week 10—completer | 18 | 12 | 20 |
| Responders | 6 (33.3) | 9 (75.0) | 8 (40.0) |
| P value | 0.723 | 0.026 | — |

The proportion of patients who achieved at least 50% reduction threshold in 7-day average daily diary-reported worst itch intensity NRS score from baseline to Week 10/LOV for each dose of nalbuphine, separately and placebo in the MITT population is presented in Table 6. For LOV patients, no statistical differences were observed although these data suggest a trend towards improvement in the nalbuphine 180 mg group compared to the placebo group. For patients who completed the study, a statistically significant difference in the proportion of responders was noted between the nalbuphine 180 mg treatment group and the placebo treatment group (P=0.028).

TABLE 6

| | | |
|---|---|---|
| Nalbuphine | Nalbuphine | |
| 90 mg | 180 mg | Placebo |
| N (%) | N (%) | N (%) |

Patients meeting responder criterion at

| | | | |
|---|---|---|---|
| Week 10—LOV | N = 22 | N = 18 | N = 22 |
| Responders | 3 (13.6%) | 6 (33.3%) | 4 (18.2%) |
| p-value | 0.981 | 0.083 | — |

Patients meeting responder criterion at

| | | | |
|---|---|---|---|
| Week 10—completer | N = 18 | N = 12 | N = 20 |
| Responders | 2 (11.1%) | 6 (50%) | 4 (20%) |
| p-value | 0.649 | 0.028 | — |

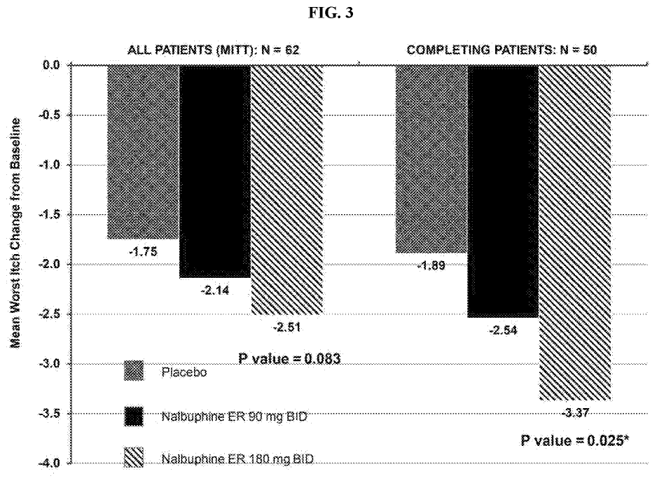

FIG. 3 is a graphical representation of Mean Change from Baseline to the Last Observation in the Worst Itching Numerical Rating Scale for all MITT patients (N=62, at left) and for completing patients (N=50, at right). For patients who completed the study, a statistically significant difference in the Mean Change from Baseline to the Last Observation in the Worst Itching Numerical Rating Scale was noted between the nalbuphine 180 mg treatment group and the placebo treatment group (P=0.025).

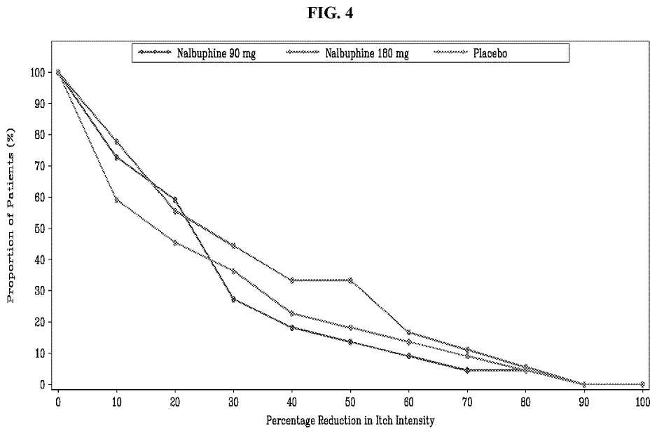

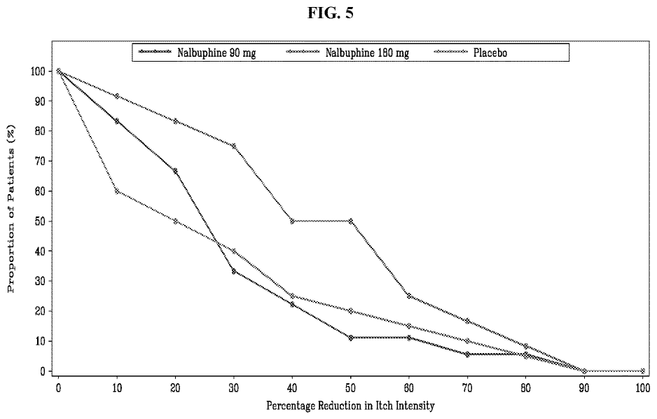

FIGS. 4 and 5 show the distribution of patients among various magnitudes of reduction in 7-Day worst itch intensity from baseline to LOV and Week 10 in the MITT population and for MITT patients who completed the study, respectively. Treatment effects owing to the nalbuphine 180 mg dose were observable beginning at the level of ≥30% reduction in the worst itch intensity, extending well through the ≥50% threshold where the distinction between the nalbuphine 180 mg was most apparent. The difference in observed response proportions between the nalbuphine 180 mg treatment group and placebo treatment group was greater and more consistent across the range of response thresholds for patients who completed the study.

The difference in change from baseline average itch intensity NRS score between the nalbuphine 180 mg treatment group and placebo treatment group was statistically significant at end of treatment (LS mean treatment difference: −1.45; 95% CI: −2.764, −0.134; P=0.031).

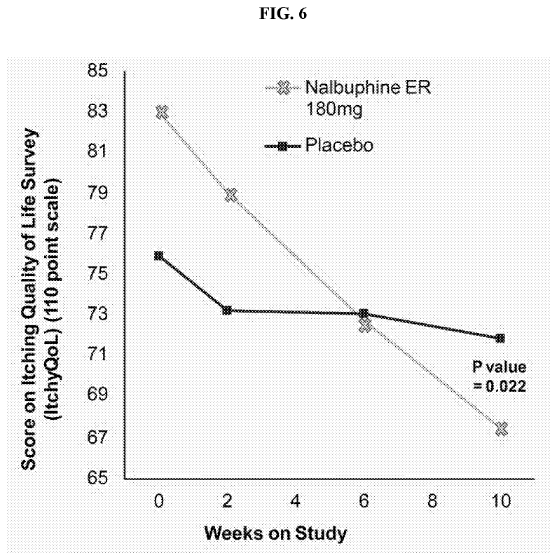

The mean ItchyQoL total score decreased at each time point from baseline in all the treatment groups. The decrease in mean ItchyQoL total score was greater for both nalbuphine treatment groups compared with the placebo treatment group. The mean (SD) decrease in ItchyQoL total score from baseline to Week 10 was −7.79 (7.83), −13.83 (13.45), and −5.45 (10.91) in the nalbuphine 90 mg treatment group, nalbuphine 180 mg treatment group, and the placebo treatment group, respectively. FIG. 6 shows the mean ItchyQoL total score for the nalbuphine 180 mg treatment group and placebo. Based on a mixed model analysis of repeated measures, the difference between the nalbuphine 180 mg treatment group and placebo was statistically significant at Week 10 (LS mean: −8.75; p=0.022).

### Conclusions

The main efficacy results of this study indicate that PN patients experienced an antipruritic effect at the 180 mg BID dose for those subjects who can tolerate the drug in the titration period and complete treatment. This is most evident when defining response as a required 50% or greater reduction in worst itch NRS intensity from baseline following 10 weeks of continuous therapy.

The total ItchyQoL score for MITT patients who completed the study showed for nalbuphine 180 mg treatment group a clinically meaningful and statistically significant mean reduction (p=0.022) at Week 10 compared to placebo. Thus, the ItchyQoL score change is supportive of a clinically meaningful change in the quality of life of the PN patients in the nalbuphine 180 mg treatment group who completed the study.

### Example 3

This clinical study was a Phase 2 Open Label Extension Study (clinical study TR03ext) of the randomized, double-blind, 3-parallel arm, placebo controlled dose ranging study described in Example 2. All those subjects who were randomized, and then completed Example 2 study, were eligible to participate in the Extension Study.

The primary endpoint was a description of the overall incidence and nature of Treatment-Emergent AEs (TEAES) during weeks 5-50 of the study. Exploratory efficacy endpoints included Change from Baseline patient reported outcome measures of Worst itch NRS, itchy VRS and ItchyQoL, by Treatment Period study visit and Baseline NRS score. There was also an assessment of any potential skin lesion changes over time using the metrics of the PAS.

### Number of Patients (Planned and Analyzed)

Of the 63 patients that were randomized to the study of Example 2, 36 subjects who enrolled into Extension Study entered the Treatment Period of the study and were exposed to nalbuphine HCl ER tablet administration and are the basis of the study safety population analysis.

### Dosing

Patients in Extension Study were titrated over a dose range of 30 mg QD to 180 mg BID based on tolerability. The selected dose range was based on Example 2 study whereby patients were titrated from a 30 mg QD dose either to 30 mg BID, 60 mg BID, 90 mg BID, 120 mg BID or a 180 mg BID dose. The highest dose proposed was 180 mg BID (360 mg daily dose), and was well below the highest recommended daily treatment of 160 mg IV (equivalent to 960 mg oral) for the current marketed parenteral administered product. Table 7 shows the dosing during the titration period, which depends on the patient's tolerance to a particular drug dose.

TABLE 7

| Week of | | | | |
|---|---|---|---|---|
| Treatment | | | | |
| Period¹ | | Drug Tolerance | | |
| (TV or TC) | Day | Acceptable | Unacceptable³ | Discontinuation |
| Week 1 | 1 | 30 mg (PM dose) | — | |
| (TV1/ | 2 | 0 mg (AM dose) | — | |
| Visit1a/1b) | | 30 mg (PM dose) | | |
| | 3-4 | 30 mg BID | | |
| | 5-7 | Titrate patients to | Reduce dose | NA |
| | | tolerability with | incrementally | |
| | | dose increases of | by 30 mg BID | |
| | | 30 mg BID | | |
| | | Maintain dose for | | |
| | | at least 3 to 4 | | |
| | | days up to next | | |
| | | dose level | | |

The highest possible titration dose by the end of Treatment Week
1 is 60 mg BID

| | | | | |
|---|---|---|---|---|
| Week 2² | 1-7 | Continue | Reduce dose | If 30 mg BID is |
| TC#1 | | incremental | incrementally | not tolerated |
| | | increase in dose | by 30 mg BID | within one |
| | | and maintaining | | week, |
| | | at each dosed | | discontinue |
| | | level for 3 to 4 | | patient |
| | | days | | |

The highest possible titration dose by the end of Treatment Week
2 is 120 mg BID

| | | | | |
|---|---|---|---|---|
| Week 3² | 1-7 | Continue dose | Reduce dose by | If 30 mg BID is |
| | | increase | 30 mg BID and | not tolerated |
| | | | maintain | within one |
| | | | | week, |
| | | | | discontinue |
| | | | | patient |

The highest possible titration dose by the end of Treatment Week
3 is 180 mg BID

| | | | | |
|---|---|---|---|---|
| Week 4² | 1-7 | Maintain dose at | Reduce dose | NA |
| (TC#2) | | highest dose | reached on | |
| | | level reached on | Week 3 Day 7 | |
| | | Week 3 Day 7 | so that subject | |
| | | | is now in | |
| | | | maintenance | |
| | | | dosing at one of | |
| | | | the following: | |
| | | | 30 mg BID, 60 | |
| | | | mg BID, 90 mg | |
| | | | BID, 120 mg | |
| | | | BID or 180 mg | |
| | | | BID | |
| Treatment | 1-7 | Maintain dose | Patient may | Patient |
| Week 5⁴ up | | from Week 4 | down titrate | discontinued if |
| to Treatment | | through TV14 | twice over | a 3^{rd} time down- |
| Week 50 | | | remaining | titration is |
| (TV3-TV14) | | | duration of | needed. |
| | | | study | |

¹The decision to enter the patient into the Titration Period is based on Worst Itch NRS score NRS ≥5 obtained from the patient on Visit 1a or 1b
²The titration decision will be made based on tolerance to study drug
³Tolerance level is unacceptable to either the patient and/or investigator
⁴The number of Treatment Weeks prior to TV14 will vary for patients previously in the Observation Period depending upon the number of weeks spent in the Observation Period.

### Study Design

The study duration for each patient was up to 52 weeks, with up to 50 weeks on study drug. The Extension Study consisted of a Treatment Period (that was followed by a Washout Safety Follow-up Period) and an Observation Period. Patients either entered directly into a drug Treatment Period (NRS >5) or a no-drug Observation Period (NRS ≤5) based on their reported NRS scores on the first Visit (Visit 1a). For up to 12 Extended Screening weeks, patients in the no-drug Observation Period could have also transitioned into the drug Treatment Period if their NRS increased to NRS >5. All patients entering the Treatment Period, whether immediately upon study entry or following a period of time in the Observation Period, were titrated to a dose ranging between 30 mg up to 180 mg BID, the highest dose tested in Example 2. Patients who completed the Observation Period and whose NRS did not exceed NRS >5 over the 12 weeks were considered screen failures.

The total study duration for any individual patient was up to 52 weeks. For patients who enter directly into the Treatment Period, the total amount of time on drug did not exceed 50 weeks. For patients who entered the Treatment Period from the Observation Period, the total amount of time spent in the combined two periods of the study could not exceed 50 weeks. All patients on drug treatment had a 2-week washout and safety follow-up period at the end of the dosing period.

The total amount of time in the Observation Period did not exceed 12 weeks. After these 12 Extended Screening weeks, subjects not eligible for the Treatment Period were screen failed from the study.

The three TR03 Extension Study periods are summarized in Table 8.

TABLE 8
Description of Study Periods

| Study Period | Study Weeks | Duration |
|---|---|---|
| Observation | Patients who did not meet the criteria to | Up to 12 |
| Period | start Treatment (NRS =< 5) were followed | Extended |
| | in Observation Period visits up to 12 | Screening |
| | weeks. | |
| | If NRS remained at or below 5 over the 12 | weeks |
| | weeks, participation in the study ended, | |
| | and the patient was screen failed. | |
| | If NRS exceeded 5 during the 12-week | |
| | observation period, the patient became | |
| | eligible to enter the Treatment Period | |
| | (defined below) | |
| Treatment | For patients directly entering the | Up to |
| Period | Treatment Period (NRS > 5) as of Visit | 50 weeks |
| | 1a, treatment Period began with Study | |
| | Week 1 (Visit 1a) and ended with Study | |
| | Week 50 | |
| | For patients entering the Treatment Period | |
| | after being followed in the Observation | |
| | Period, the number of weeks on treatment | |
| | was equal to 50 weeks minus weeks in | |
| | Observation period. As a result, the end of | |
| | the Treatment Period varied. | |
| | The End of Treatment Visit took place | |
| | after the patient completed the last week | |
| | of study drug | |
| Washout | The Washout and Safety Follow-up | 2 weeks |
| and Safety | Period was two (2) weeks in duration. | |
| Follow-Up | For patients directly entering the | |
| Period | Treatment Period as of Visit 1a, and | |
| | completing 50 weeks of study drug | |
| | treatment, the Washout and Safety | |
| | Follow-up Period took place during weeks | |
| | 51 and 52. | |
| | For patients entering the Treatment Period | |
| | after being followed in the Observation | |
| | Period, the Washout and Safety Follow-up | |
| | Period took place during the two (2) | |
| | weeks after they completed the last week | |
| | of study drug. | |
| | For all patients, the final Visit was | |
| | scheduled within a week from completion | |
| | of the Washout and Safety Follow-up | |
| | Period. | |

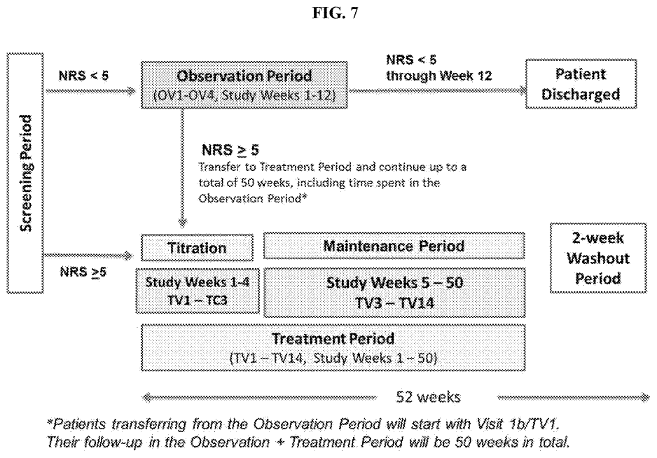

An overall study schematic is shown in FIG. 7.

### Results

Table 9 displays the mean reduction from baseline in worst-itch NRS and VRS by study week. The worst-itch mean (SD) score and itchy VRS mean (SD) score continued to diminish over time in the study population.

TABLE 9

| | Worst Itch NRS | | | VRS Itch | | |
|---|---|---|---|---|---|---|
| | | Visit | Mean (SD) | | Visit | Mean (SD) |
| Study | | Mean | Change from | | Mean | Change from |
| Week | N | (SD) | Baseline | N | (SD) | Baseline |
| Baseline | 36 | 7.6 (1.6) | | 36 | 2.7 (0.8) | |
| 3 | 34 | 5.7 (2.4) | −2.0 (2.2) | 34 | 2.1 (1.1) | −0.6 (0.9) |
| 5 | 33 | 5.2 (2.5) | −2.5 (2.2) | 33 | 1.8 (1.0) | −0.9 (1.0) |
| 9 | 30 | 4.6 (2.3) | −2.9 (2.2) | 30 | 1.7 (0.9) | −0.9 (0.8) |
| 13 | 25 | 4.8 (2.2) | −2.7 (1.8) | 25 | 1.5 (0.8) | −1.1 (0.8) |
| 17 | 25 | 3.8 (2.4) | −3.6 (2.6) | 25 | 1.4 (1.0) | −1.2 (1.2) |
| 21 | 22 | 4.1 (2.4) | −3.3 (2.4) | 22 | 1.4 (0.7) | −1.2 (0.8) |
| 26 | 21 | 3.4 (1.6) | −3.9 (2.2) | 21 | 1.4 (0.9) | −1.2 (0.9) |
| 30 | 20 | 3.2 (1.6) | −4.2 (2.3) | 20 | 1.1 (0.8) | −1.5 (0.8) |
| 34 | 18 | 3.3 (1.6) | −3.9 (2.1) | 18 | 1.4 (0.6) | −1.2 (0.8) |
| 38 | 17 | 3.2 (1.6) | −3.9 (1.7) | 17 | 1.4 (0.6) | −1.2 (0.7) |
| 42 | 17 | 3.2 (1.4) | −3.9 (1.4) | 17 | 1.2 (0.6) | −1.4 (0.6) |
| 46 | 17 | 3.2 (1.5) | −3.9 (1.3) | 17 | 1.2 (0.7) | −1.4 (0.6) |
| 50 | 15 | 2.9 (1.7) | −4.3 (1.3) | 15 | 1.1 (0.6) | −1.5 (0.7) |

Table 10 displays the distribution of itchy Verbal Rating Score (VRS) from baseline to last observed value by change of category. A large majority of subjects reported at least one category improvement from baseline to last observed value.

TABLE 10

| | | Patients | Patients | |
|---|---|---|---|---|
| | | With At | With At | |
| | | Least 6 | Least 9 | |
| VRS Itch | All | Months of | Months of | |
| Improvement | Patients | Treatment | Treatment | Completers |
| Category | N = 36 | N = 18 | N = 15 | N = 14 |
| 4 Grades Better | 0 | 0 | 0 | 0 |
| 3 Grades Better | 1 (2.8%) | 1 (5.6%) | 0 | 0 |
| 2 Grades Better | 9 (25.0%) | 9 (50.0%) | 7 (46.7%) | 7 (50.0%) |
| 1 Grade Better | 16 (44.4%) | 6 (33.3%) | 6 (40.0%) | 5 (35.7%) |
| No Change | 3 (8.3%) | 2 (11.1%) | 2 (13.3%) | 2 (14.3%) |
| 1 Grade Worse | 0 | 0 | 0 | 0 |
| 2-4 Grades | 0 | 0 | 0 | 0 |
| Worse | | | | |

Table 11 displays the mean reduction from baseline in ItchyQoL Total Score by study week. The mean ItchyQoL total score in the open label study population was decreased from the baseline value at every visit throughout the 50 weeks of drug treatment.

TABLE 11

| | ItchyQoL Total Score | | |
|---|---|---|---|
| | | Visit | Mean (SD) Change |
| Study Week | N | Mean (SD) | from Baseline |
| Baseline | 34 | 75.2 (15.1) | |
| 3 | 33 | 70.3 (18.7) | −5.3 (11.2) |
| 5 | 31 | 67.8 (19.0) | −7.6 (14.8) |
| 9 | 28 | 65.8 (18.5) | −9.8 (14.8) |
| 13 | 24 | 65.7 (17.4) | −10.2 (15.9) |
| 17 | 24 | 63.5 (19.4) | −12.9 (17.0) |
| 21 | 21 | 62.6 (18.0) | −13.5 (13.4) |
| 26 | 20 | 59.4 (16.8) | −16.0 (15.0) |
| 30 | 19 | 60.8 (18.9) | −14.6 (14.2) |
| 34 | 17 | 62.2 (15.2) | −13.5 (10.8) |
| 38 | 16 | 61.9 (16.1) | −13.3 (13.6) |
| 42 | 16 | 63.4 (15.3) | −11.8 (10.0) |
| 46 | 16 | 62.4 (14.1) | −12.8 (9.7) |
| 50 | 14 | 63.3 (15.9) | −13.2 (11.0) |

Tables 12, 13 and 14 display healed lesion PAS data for subjects who received treatment for at least 6 months, subjects who received treatment for at least 9 months and subjects who completed 50 weeks of treatment in the study, respectively. The status of the healed lesion activity is reported as percentages based on their stage (0-4) using a grading system: Stage 0 (100% healed); Stage 1 (75%-99% healed); Stage 2 (50%-74% healed); Stage 3 (25%-49% healed); Stage 4: 0-24% healed. Thus the higher the stage, the more healed the prurigo skin pathology. The data in Tables 12, 13 and 14 show that many subjects showed at least one category improvement in the percentage of healed lesions (subjects who improved in the shift tables are boldfaced) after 6 (10/16; 62.5%), 9 (7/13; 53.8%) and 12 months (6/12; 50%).

TABLE 12
Healed lesion PAS data for subjects who
received treatment for at least 6 months

| | Baseline PAS Result | | | | |
|---|---|---|---|---|---|
| | Stage/Number (%) of Subjects | | | | |
| Last Observed | Stage 0 | Stage 1 | Stage 2 | Stage 3 | Stage 4 |
| PAS Result | 1 (5.9) | 3 (17.6) | 4 (28.6) | 3 (21.4) | 6 (35.3) |
| Stage/Number | PAS Category Shift | | | | |
| (%) of Subjects | Number (%) of Subjects | | | | |
| Stage 0 | 1 (5.9) | 0 | 1 (5.9) | 0 | 1 (5.9) |
| 3 (17.6) | | | | | |
| Stage 1 | 0 | 2 (11.8) | 3 (17.6) | 1 (5.9) | 2 (11.8) |
| 9 (52.9) | | | | | |
| Stage 2 | 0 | 1 (5.9) | 0 | 0 | 1 (5.9) |
| 2 (11.8) | | | | | |
| Stage 3 | 0 | 0 | 0 | 2 (11.8) | 1 (5.9) |
| 3 (17.6) | | | | | |
| Stage 4 | 0 | 0 | 0 | 0 | 1 (5.9) |
| 1 (5.9) | | | | | |

TABLE 13
Healed lesion PAS data for subjects who
received treatment for at least 9 months

| | Baseline PAS Result | | | | |
|---|---|---|---|---|---|
| | Stage/Number(%) of Subjects | | | | |
| Last Observed | Stage 0 | Stage 1 | Stage 2 | Stage 3 | Stage 4 |
| PAS Result | 1 (7.1) | 3 (21.4) | 3 (21.4) | 2 (14.3) | 5 (35.7) |
| Stage/Number | PAS Category Shift | | | | |
| (%) of Subjects | Number (%) of Subjects | | | | |
| Stage 0 | 1 (7.1%) | 0 | 0 | 0 | 1 (7.1) |
| 2 (14.3) | | | | | |
| Stage 1 | 0 | 2 (14.3) | 3 (21.4) | 0 | 1 (7.1) |
| 6 (42.9) | | | | | |
| Stage 2 | 0 | 1 (7.1) | 0 | 0 | 1 (7.1) |
| 2 (14.3) | | | | | |
| Stage 3 | 0 | 0 | 0 | 2 (14.3) | 1 (7.1) |
| 3 (21.4) | | | | | |
| Stage 4 | 0 | 0 | 0 | 0 | 1 (7.1) |
| 1 (7.1) | | | | | |

TABLE 14
Healed lesion PAS data for subjects
who received treatment for 50 weeks

| | Baseline PAS Result | | | | |
|---|---|---|---|---|---|
| | Stage/Number (%) of Subjects | | | | |
| Last Observed | Stage 0 | Stage 1 | Stage 2 | Stage 3 | Stage 4 |
| PAS Result | 1 (7.7) | 3 (23.1) | 3 (23.1) | 2 (15.4) | 4 (30.8) |
| Stage/Number | PAS Category Shift | | | | |
| (%) of Subjects | Number (%) of Subjects | | | | |
| Stage 0 | 1 (7.7) | 0 | 0 | 0 | 1 (7.7) |
| 2 (15.4) | | | | | |
| Stage 1 | 0 | 2 (15.4) | 3 (23.1) | 0 | 0 (7.7) |
| 6 (46.1) | | | | | |
| Stage 2 | 0 | 1 (7.7) | 0 | 0 | 1 (7.7) |
| 2 (15.4) | | | | | |
| Stage 3 | 0 | 0 | 0 | 2 (15.4) | 1 (7.7) |
| 3 (23.1) | | | | | |
| Stage 4 | 0 | 0 | 0 | 0 | 1 (7.7%) |
| 1 (7.7) | | | | | |

Table 15, 16 and 17 display excoriations/crusts lesion PAS data for subjects who received treatment for at least 6 months, subjects who received treatment for at least 9 months and subjects who completed 50 weeks of treatment in the study, respectively. The status of the prurigo lesions with excoriations/crusts is reported as percentages based on their stage (0-4) using a grading system: Stage 0 (0% excoriations/crusts); Stage 1 (10%-25% excoriations/crusts); Stage 2 (26%-50% excornations/crusts); Stage 3 (51%-75% excoriations/crusts); Stage 4: (76%-100% excoriations/crusts). Thus, the higher the stage, the more severe is the prurigo skin pathology. The data in Table 15, 16 and 17 show that many subjects showed at least one category improvement (lesser stage) in the percentage of excoriations/crust lesions after 6 months (13/16; 81.2%), 9 months (9/13; 69.2%), and 12 months (8/12; 66.60%), respectively (subjects who improved in the shift tables are boldfaced). Table 15: Excoriated/Crusts PAS data for subjects who received treatment for at least 6 months

TABLE 15
Excoriated/Crusts PAS data for subjects who
received treatment for at least 6 months

| Last | Baseline PAS Result | | | | |
|---|---|---|---|---|---|
| Observed | Stage/Number (%) of Subjects | | | | |
| PAS Result | Stage 0 | Stage 1 | Stage 2 | Stage 3 | Stage 4 |
| Stage/ | 1 (5.9) | 2 (11.8) | 6(35.3) | 4 (23.5) | 4 (23.5) |
| Number | | | | | |
| (%) of | PAS Category Shift | | | | |
| Subjects | Number (%) of Subjects | | | | |
| Stage 0 | 1 (5.9%) | 0 | 1 (5.9%) | 0 | 1 (5.9%) |
| 3 (17.6) | | | | | |
| Stage 1 | 0 | 1 (5.9%) | 5 (29.4%) | 1 (5.9%) | 2 (11.8%) |
| 9 (52.9) | | | | | |
| Stage 2 | 0 | 0 | 0 | 2 (11.8%) | 0 |
| 2 (11.8) | | | | | |
| Stage 3 | 0 | 0 | 0 | 1 (5.9%) | 0 |
| 1 (5.9) | | | | | |
| Stage 4 | 0 | 1 (5.9%) | 0 | 0 | 1 (5.9%) |
| 2 (11.8) | | | | | |

TABLE 16
Excoriated/Crusts PAS data for subjects who
received treatment for at least 9 months

| | Baseline PAS Result | | | | |
|---|---|---|---|---|---|
| | Stage/Number (%) of Subjects) | | | | |
| Last Observed | Stage 0 | Stage 1 | Stage 2 | Stage 3 | Stage 4 |
| PAS Result | 1 (7.1) | 2(14.3) | 5(35.7) | 3 (21.4) | 3 (21.4) |
| Stage/Number | PAS Category Shift | | | | |
| (%) of Subjects | Number (%) of Subjects | | | | |
| Stage 0 | 1 (7.1%) | 0 | 0 | 0 | 1 (7.1%) |
| 2 (14.3) | | | | | |
| Stage 1 | 0 | 1 (7.1%) | 5 (35.7%) | 0 | 1 (7.1%) |
| 7 (50.0) | | | | | |
| Stage 2 | 0 | 0 | 0 | 2 (14.3%) | 0 |
| 2 (14.3) | | | | | |
| Stage 3 | 0 | 0 | 0 | 1 (7.1%) | 0 |
| 1 (7.1) | | | | | |
| Stage 4 | 0 | 1 (7.1%) | 0 | 0 | 1 (7.1%) |
| 2 (14.3) | | | | | |

TABLE 17
Excoriated/Crusts PAS data for subjects who received
treatment for 50 weeks

| | Baseline PAS Result | | | | |
|---|---|---|---|---|---|
| | Stage/Number (%) of Subjects | | | | |
| Last Observed | Stage 0 | Stage 1 | Stage 2 | Stage 3 | Stage 4 |
| PAS Result | 1 (7.7) | 2 (15.4) | 5 (38.5) | 3 (23.1) | 2 (15.4) |
| Stage/Number | PAS Category Shift | | | | |
| (%) of Subjects | Number (%) of Subjects | | | | |
| Stage 0 | 1 (7.1%) | 0 | 0 | 0 | 1 (7.7%) |
| 2 (15.4) | | | | | |
| Stage 1 | 0 | 1 (7.7%) | 5 (38.5%) | 0 | 0 |
| 6 (46.2) | | | | | |
| Stage 2 | 0 | 0 | 0 | 2 (15.4%) | 0 |
| 2 (15.4) | | | | | |
| Stage 3 | 0 | 0 | 0 | 1 (7.7%) | 0 |
| 1 (7.7) | | | | | |
| Stage 4 | 0 | 1 (7.7%) | 0 | 0 | 1 (7.7%) |
| 2 (15.4) | | | | | |

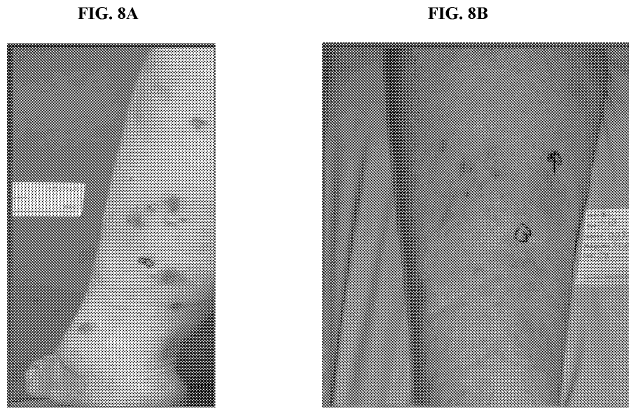

FIG. 8A shows the pruriginous lesions of a patient from Example 2 at baseline and FIG. 8B shows the healed pruriginous lesions of the same patient at Week 50 in the extension study described in Example 3 (TR03ext).

### CONCLUSIONS

The open label extension long-term NRS worst-itch data and VRS summarized in Table 9 are consistent with the results of the parent double-blinded study anti-pruritic efficacy results summarized in Tables 5 and 6. This conclusion is supported by the VRS open label data results (Table 10), which show that all the subjects reported a sustained improvement of at least 1-category when treated for at least 6 months or more on study drug. The ItchyQoL instrument data is also supportive of improvement in the worst-itch NRS findings (Table 11).

The PAS excoriative lesion data shown in Tables 15, 16 and 17 and healed prurigo lesion data shown in Tables 12, 13 and 14 indicate that there is clinical evidence that the itch-scratch cycle may been beneficially altered in many subjects by nalbuphine HCl ER treatment leading to improvement of prurigo lesions over longer durations (≥6 months) of treatment.

What is claimed is:

1. A method of treating prurigo nodularis, comprising:
(a) a titration period, comprising administering nalbuphine or a pharmaceutically acceptable salt or ester thereof to a patient in need thereof according to the following dose schedule:

| Day | AM dosage (mg) | PM dosage (mg) |
|---|---|---|
| Day 1 | 0 | 30 |
| Day 2 | 0 | 30 |
| Day 3 | 30 | 30 |
| Day 4 | 30 | 30 |
| Day 5 | 30 | 60 |
| Day 6 | 60 | 60 |
| Day 7 | 60 | 60 |
| Day 8 | 60 | 90 |
| Day 9 | 90 | 90 |
| Day 10 | 90 | 90 |
| Day 11 | 90 | 120 |
| Day 12 | 120 | 120 |
| Day 13 | 120 | 120 |
| Day 14 | 120 | 180 |

and
(b) a treatment period, comprising administering a daily dose of about 360 mg of nalbuphine or a pharmaceutically acceptable salt or ester thereof to the patient for at least a week following the titration period.

2. The method of claim 1, wherein the treatment period comprises administering about 180 mg of nalbuphine or a pharmaceutically acceptable salt or ester thereof twice a day.

3. The method of claim 1, wherein the treatment period comprises administering about 360 mg of nalbuphine or a pharmaceutically acceptable salt or ester thereof once a day.

4. The method of claim 1, wherein the treatment period is for at least 3 weeks.

5. The method of claim 1, wherein the treatment period is for at least 10 weeks.

6. The method of claim 1, wherein the treatment period is for about 3 weeks to 50 weeks.

7. The method of claim 1, wherein the patient has mild, moderate, or severe prurigo nodularis.

8. The method of claim 1, wherein the patient has moderate or severe prurigo nodularis.

9. The method of claim 1, wherein the nalbuphine or a pharmaceutically acceptable salt or ester thereof is nalbuphine hydrochloride.

10. The method of claim 1, wherein the nalbuphine or a pharmaceutically acceptable salt or ester thereof is in the form of an extended release oral dosage form.

11. The method of claim 10, wherein the extended release oral dosage form comprises a pharmaceutical diluent, a hydrophilic compound and a cross-linking agent.

12. The method of claim 10, wherein the extended release oral dosage form comprises about 8% to about 30% by weight of a hydrophilic compound and about 5% to about 20% by weight of a cross-linking agent.

13. The method of claim 12, wherein the cross-linking agent is calcium sulfate dihydrate.

14. The method of claim 12, wherein the hydrophilic compound is locust bean gum, xanthan gum, or a mixture thereof.

15. The method of claim 10, wherein the extended release oral dosage form comprises nalbuphine hydrochloride, mannitol, hydroxypropyl cellulose, locust bean gum, xanthan gum, calcium sulfate dihydrate, and magnesium stearate.

16. The method of claim 10, wherein the extended release oral dosage form comprises about 15% to about 25% by weight of locust bean gum, about 10% to about 20% by weight of xanthan gum, about 50% to about 85% by weight of mannitol and about 5% to 15% by weight of calcium sulfate dihydrate.

17. The method of claim 1, wherein after the treatment the patient experiences a reduction of itch that is characterized by at least a 30% decline in worst itching intensity Numerical Rating Scale (NRS) value.

18. The method of claim 1, wherein after the treatment the patient experiences a reduction of itch that is characterized by at least a 50% decline in worst itching intensity NRS value.

19. The method of claim 1, wherein after the treatment the patient experiences a reduction of itch that is characterized by at least a 4 point reduction in worst itching intensity NRS value.

20. The method of claim 1, wherein after said treating the patient experiences a reduction of itch that is characterized by at least a 10% improvement in Itchy Quality of Life (ItchyQoL) scale.

21. The method of claim 20, wherein the patient experiences a reduction of itch that is characterized by at least a 10% improvement in the ItchyQoL symptoms subscale.

22. The method of claim 20, wherein the patient experiences a reduction of itch that is characterized by at least a 10% improvement in the ItchyQoL functional subscale.

23. The method of claim 20, wherein the patient experiences a reduction of itch that is characterized by at least a 10% improvement in the ItchyQoL emotion subscale.

24. The method of claim 1, wherein after said treating the patient experiences a reduction of itch that is characterized by at least a one category/stage improvement in Prurigo Activity Score (PAS) scale in the domains of number of prurigo lesions, prurigo lesions with excoriations or crusts, or healed prurigo lesions.

25. The method of claim 24, wherein the patient experiences a reduction of itch that is characterized by at least a one stage improvement in PAS staging of pruriginous lesions with excoriations or crusts.

26. The method of claim 24, wherein the patient experiences a reduction of itch that is characterized by at least a one stage improvement in PAS staging of healed lesions.

27. The method of claim 24, wherein the patient experiences a reduction of itch that is characterized by at least a one category improvement in the number of prurigo lesions.

28. The method of claim 1, wherein after said treating the patient experiences healing of pruriginous lesions.

29. The method of claim 28, wherein the pruriginous lesions are selected from nodules, papules or plaques.

30. The method of claim 1, wherein after said treating the patient experiences a reduction in lesions with excoriations or crusts.
